(19) United States
(12) Patent Application Publication
(10) Pub. No.: US 2006/0002964 A9
(43) Pub. Date: Jan. 5, 2006

(54) Crosslinked cosmetic or pharmaceutical phospholipid-containing gels and emulsions based on ethylene oxide-containing or propylene oxide-containing emulsifiers

(75) Inventors: Jorg Schreiber, Hamburg (DE); Mirko Tesch, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/953,588

(22) Filed: Sep. 28, 2004

Related U.S. Application Data
(63) Continuation of application No. PCT/EP03/03166, filed on Mar. 27, 2003.

(30) Foreign Application Priority Data
Mar. 28, 2002 (DE) 102 13 957.1

(51) Int. Cl. A61K 7/00

(52) U.S. Cl. 424/401

(57) ABSTRACT

The invention is a process for making a cosmetic or pharmaceutical, phospholipid-containing crosslinked microemulsion gel or crosslinked oil-in-water emulsion. The process includes the use of at least one crosslinker that is added to at least one of the water phase and the oil phase of the emulsion, or the is added after the water phase and oil phase are mixed.

### CROSS-REFERENCE TO RELATED APPLICATIONS

This is a continuation application of PCT/EP03/03166, filed Mar. 27, 2003, which is incorporated herein by reference in its entirety, and also claims the benefit of German Priority Application No. 102 13 957.1, filed Mar. 28, 2002.

### FIELD OF THE INVENTION

The present invention relates to crosslinked phospholipid-containing microemulsion gels and emulsions of the oil-in-water type, to processes for their preparation, and to their use for cosmetic or pharmaceutical purposes. In particular, they are applied topically.

### BACKGROUND OF THE INVENTION

Cosmetic skincare is primarily understood as meaning that the natural function of the skin as a barrier against environmental influences (e.g. dirt, chemicals and microorganisms) and against the loss of substances intrinsic to the body (e.g. water, natural fats and electrolytes) is strengthened or restored.

Impairment of this function may lead to increased absorption of toxic or allergenic substances or to attack by microorganisms, leading to toxic or allergic skin reactions.

Another aim of skincare is to compensate for the loss by the skin of lipids and water caused by daily washing. This is particularly important when the natural regeneration ability is inadequate. Furthermore, skincare products should protect against environmental influences, in particular against sun and wind, and delay skin aging.

Medicinal topical compositions generally comprise one or more medicaments in an effective concentration. For the sake of simplicity, for a clear distinction between cosmetic and medicinal use and corresponding products, reference is made to the legal provisions of the Federal Republic of Germany (e.g. Cosmetics Directive, Foods and Drugs Act).

Customary cosmetic and dermatological preparation forms which have become ever more widespread in recent times are gels.

In the technical sense, gels are understood as meaning: relatively dimensionally stable, readily deformable disperse systems of at least two components, which usually consist of an—in most cases solid—colloidally dispersed substance of long-chain molecule groups (e.g. gelatin, silica and polysaccharides) as structure former and a liquid dispersant (e.g. water). The colloidally dispersed substance is often referred to as a thickener or gelling agent. It forms a spatial network within the dispersant, where individual colloidal particles may be joined together with greater or lesser strength via electrostatic interaction. The dispersant which surrounds the network is characterized by electrostatic affinity to the gelling agent, i.e. a predominantly polar (in particular: hydrophilic) gelling agent preferentially gels a polar dispersant (in particular: water), whereas a predominantly nonpolar gelling agent preferentially gels nonpolar dispersants.

Strong electrostatic interactions which are realized, for example, in hydrogen bridge bonds between gelling agent and dispersant, but also between dispersant molecules with one another, can lead to considerable crosslinking also of the dispersant. Hydrogels can consist of virtually 100% of water (besides, for example, about 0.2-1.0% of a gelling agent) and have an entirely solid consistency. The water content is present here in ice-like structural elements, meaning that gels therefore do justice to the origin of their name [from Latin “gelatum”=“frozen” via the alchemistic term “gelatina” (16^{th} century) for the modern term “gelatin”].

In cosmetics and pharmaceutical technology, lipogels and oleogels (of waxes, fats and fatty oils), and also carbogels (of paraffin or petrolatum) are also common. In practice, a distinction is made between oleogels, which are virtually free from water, and hydrogels, which are virtually free from fat. In most cases, gels are transparent. In cosmetics or pharmaceutical technology, gels are usually characterized by a semisolid, often flowable consistency.

In simple emulsions, finely disperse droplets of one phase (water droplets in W/O emulsions or lipid vesicles in O/W emulsions), surrounded by an emulsifier shell, are present in a second phase. The droplet diameters of customary emulsions are in the range from about 200 nm to about 50 μm. Such “macroemulsions” are, without further coloring additives, milky white in color and opaque. Only micellar and molecular solutions having particle diameters of less than about 10⁻² μm appear clear and transparent.

In addition, so-called surfactant gels are customary preparations of the prior art. This is understood as meaning systems which, besides water, have a high concentration of emulsifiers, typically more than about 25% by weight, based on the overall composition. If oil components are solubilized in these surfactant gels, microemulsion gels are obtained which are also referred to as “ringing gels”. By adding nonionic emulsifiers, for example alkyl polyglycosides, it is possible to obtain cosmetically more elegant microemulsion gels. Here too, the high content of emulsifiers is disadvantageous.

The droplet diameter of transparent or translucent microemulsions, by contrast, is in the range from about 10 nm to 200 nm. Such microemulsions are of low viscosity or gel-like. The viscosity of many microemulsions of the O/W type is comparable with that of water.

An advantage of microemulsion gels is that active ingredients can be present in finely disperse form within the disperse phase. It is a further advantage that they may be sprayable. If microemulsions are used as cosmetics, corresponding products are characterized by high cosmetic elegance.

A disadvantage of microemulsions, and thus also of the microemulsion gels, of the prior art is that a high content of one or more emulsifiers must always be used since the small droplet size brings about a large interface between the phases, which usually has to be stabilized by emulsifiers.

The use of customary cosmetic emulsifiers is in itself safe. Nevertheless, emulsifiers, like ultimately any chemical substance, may in individual cases cause allergic reactions or reactions based on user hypersensitivity.

For example, it is known that certain photodermatoses are triggered by certain emulsifiers, but also by various fats, and simultaneous exposure to sunlight. Such photodermatoses are also called “Mallorca acne”.

### SUMMARY OF THE INVENTION

One object of the present invention was therefore to develop sunscreen products.

Thus, the present invention relates, as particular embodiments, to cosmetic and dermatological photoprotective preparations, in particular skincare cosmetic and dermatological photoprotective preparations.

The harmful effect of the ultraviolet part of solar radiation on the skin is generally known. While rays with a wavelength of less than 290 nm (the so-called UVC region) are absorbed by the ozone layer in the earth's atmosphere, rays in the range between 290 nm and 320 nm, the so-called UVB region, cause erythema, simple sunburn or even burns of greater or lesser severity.

The erythema activity maximum of sunlight is generally stated as the relatively narrow range around 308 nm.

Numerous compounds are known for protecting against UVB radiation; these are mostly derivatives of 3-benzylidenecamphor, of 4-aminobenzoic acid, of cinnamic acid, of salicylic acid, of benzophenone and also of 2-phenylbenzimidazole.

For the range between 320 nm and about 400 nm, the so-called UVA region, it is also important to have available filter substances since rays of that region can also cause damage. For example, it has been proven that UVA radiation leads to damage of the elastic and collagenous fibers of connective tissue, causing premature aging of the skin, and that it is to be regarded as a cause of numerous phototoxic and photoallergic reactions. The harmful effect of UVB radiation can be intensified by UVA radiation.

UV radiation can, however, also lead to photochemical reactions, in which case the photochemical reaction products then intervene in the skin's metabolism.

In order to prevent these reactions, antioxidants or free-radical scavengers can additionally be incorporated into the cosmetic or dermatological formulations.

Most of the inorganic pigments which are known for use in cosmetics for protecting the skin against UV rays are UV absorbers or UV reflectors. These pigments are oxides of titanium, zinc, iron, zirconium, silicon, manganese, aluminum, cerium and mixtures thereof, and also modifications.

Microemulsion gels are also suitable for other cosmetic dermatological applications, for example deodorants, meaning that the present invention relates, in a particular embodiment, to microemulsion gels as a basis for cosmetic deodorants.

Cosmetic deodorants serve to eliminate body odor which arises when fresh perspiration, which is in itself odorless, is decomposed by microorganisms. Customary cosmetic deodorants are based on different active principles.

In so-called antiperspirants, the formation of perspiration can be reduced by astringents—chiefly aluminum salts such as aluminum hydroxychloride (aluminum chlorohydrate).

By using antimicrobial substances in cosmetic deodorants it is possible to reduce the bacterial flora on the skin. In an ideal case, only the odor-causing microorganisms would be effectively reduced. The flow of perspiration itself is not influenced by this, and in an ideal case only microbial decomposition of the perspiration is temporarily stopped.

The combination of astringents with antimicrobial substances in one and the same composition is also customary.

Deodorants should satisfy the following conditions:

1) They should effect reliable deodorization.

2) The natural biological processes of the skin must not be impaired by the deodorants.

3) The deodorants must be harmless in the event of an overdose or other use which is not in accordance with the directions.

4) They should not become concentrated on the skin following repeated application.

5) They should be easy to incorporate into customary cosmetic formulations.

Liquid deodorants, for example aerosol sprays, roll-ons and the like, and also solid preparations, for example deodorant sticks, powders, powder sprays, intimate cleansing compositions etc. are known and customary.

The use of microemulsions or emulsions as bases for deodorizing or antiperspirant preparations are also known. Their relatively high content of emulsifiers, together with the described disadvantages, has hitherto been a shortcoming which is in need of remedying.

A further object of the present invention was therefore to develop preparations which are suitable as bases for cosmetic deodorants or antiperspirants and do not have the disadvantages of the prior art.

It was also an object of the invention to develop cosmetic bases for cosmetic deodorants which are characterized by good skin compatibility.

In addition, it was an object of the present invention to make available products based on crosslinked microemulsion gels or emulsions having the broadest possible application diversity. For example, bases for preparation forms such as cleansing products, face care and body care preparations were to be provided, but also decidedly medicinal-pharmaceutical administration forms, for example preparations against acne and other skin phenomena.

In a particular embodiment, the invention therefore relates to face cleansing products, preferably make-up removers, for example eye make-up removers or face care and body care preparations, make-up removal products, cleansing preparations, sunscreen products, antiwrinkle products, hair preparations, repellant products, aftersun preparations, shower bases, aftershave products, shaving preparations, deodorant/antiperspirant products, antiacne preparations.

Water-resistant eye make-up, for example mascara, can only be removed satisfactorily with aqueous-based make-up removers containing specific surfactants. However, these surfactants often only have limited physiological compatibility. When such substances come into contact with the mucous membrane, in particular the mucous membrane of the eye, they lead to irritations which manifest themselves, for example, in a reddening of the eyes. Reactions of this type are typical of surfactant-containing products.

An object of the present invention was therefore to remedy such problems.

In a further embodiment, the present invention relates to hair cosmetic preparations. In particular, the present invention relates to hair cosmetic preparations for the care of hair and the scalp. In a preferred embodiment, the present invention relates to preparations which serve to strengthen individual hairs and/or impart hold and body to the hairstyle overall.

Roughly speaking, human hair can be divided into the living part, the hair root, and the dead part, the hair shaft. The hair shaft in turn comprises the medulla which, however, as result of evolution, has become insignificant for modern man and has receded, and in cases of thin hair is often entirely absent, and also the cortex surrounding the medulla and the cuticula which encloses the totality of medulla and cortex.

The cuticula in particular, but also the keratinous region between the cuticula and cortex, as the outer sheath of the hair, are exposed to particular demands as a result of environmental influences, as a result of combing and brushing, but also as a result of hair treatment, in particular hair coloring and hair shaping, e.g. permanent waving processes.

If the stress is particularly aggressive, for example bleaching with oxidizing agents such as hydrogen peroxide, in which the pigments distributed within the cortex are destroyed by oxidation, the inside of the hair can also be affected. If human hair is to be colored permanently, in practice only oxidizing hair coloring processes are suitable. During the oxidative coloring of hair, the dye chromophores are formed as a result of the reaction of precursors (phenols, aminophenols, and less frequently also diamines) and bases (in most cases p-phenylenediamine) with the oxidizing agent, in most cases hydrogen peroxide. Hydrogen peroxide concentrations of about 6% are usually used for this.

It is usually assumed that besides the coloring action, a bleaching action also takes place as a result of the hydrogen peroxide. In oxidatively colored human hair, as in the case of bleached hair, microscopic holes are detectable at the points where melanin granules were present. The fact is that the oxidizing agent hydrogen peroxide can react not only with the dye precursors, but also with the hair substance and as a result can cause damage to the hair under certain circumstances.

Washing the hair with aggressive surfactants can also stress the hair, and at least reduce its appearance or the appearance of the hairstyle overall. For example, certain water-soluble constituents of the hair (e.g. urea, uric acid, xanthine, keratin, glycogen, citric acid, lactic acid) can be leached out as result of hair washing.

For these reasons, some hair care cosmetics which are intended to be rinsed out of the hair again once they have acted, and some of those which are to remain on the hair have been used for a relatively long time. The latter can be formulated such that they not only serve to care for the individual hairs, but also improve the appearance of the hairstyle overall, for example by imparting more body to the hair, fixing the hairstyle over a longer period or improving its ease of styling.

By using quaternary ammonium compounds, for example, the combability of the hair can be decisively improved. Such compounds attach to the hair and are often still detectable on the hair after the hair has been washed a number of times.

However, the prior art has lacked active ingredients and preparations which satisfactorily care for damaged hair. Preparations which were intended to give body to the hairstyle have also often proven to be inadequate, or they were at least unsuitable for use as hair care preparations. The hairstyle-fixing preparations of the prior art generally comprise, for example, viscous constituents, which run the risk of giving rise to a feeling of stickiness, which often has to be compensated for by skillful formulation.

An object was therefore also to overcome these disadvantages of the prior art.

A particular object of the present invention was to make available gel-like or emulsion-like preparations based on finely disperse, lecithin-containing systems of the oil-in-water type with the lowest possible emulsifier content which do not have the disadvantages of the prior art and which can be used for a very wide variety of cosmetic and/or dermatological applications, for example the uses described above. A further object of the invention was to enrich the limited range of transparent/translucent gel-like preparations based on finely disperse, lecithin-containing systems of the oil-in-water type of the prior art.

Processes for the preparation of O/W microemulsion gels by crosslinking hydrophobically modified water-soluble polymers have been described in WO 9628132. The advantageous use of lecithin was not mentioned.

Lecithin-containing microemulsions for cosmetic, pharmaceutical, parenteral applications are known from the literature and patents (see WO 9815255). It is explained therein how lecithin-containing microemulsions based on ethylene oxide-free emulsifiers can be crosslinked to give the corresponding gels. The crosslinking of lecithin-containing O/W microemulsions or O/W macroemulsions which contain ethoxylated or propoxylated O/W emulsifiers is not described. Neither is it described that the crosslinking of lecithin-containing O/W microemulsions or emulsions which contain ethoxylated or propoxylated O/W emulsifiers in combination with W/O emulsifiers is possible.

WO 0037042 describes gels which, besides lecithin, comprise an O/W and a W/O emulsifier, and water. Crosslinking with hydrophobically modified water-soluble polymers to give the microemulsion gel is not described. Lecithin organogels are described in the literature. These organogels are obtained by adding small amounts of water to a mixture of organic solvent and lecithin. Here, when water is added, inverse micelles form cylinder-like water-filled micelles (“wormlike micelles”), which become entangled with one another and thus explain the high viscosity of these mixtures. (Colloid Polym. Sci. 268, 1990, 356).

Strictly speaking, these lecithin gels do not represent microemulsion gels since the dispersed phase is not present in droplet form and additionally there is no corresponding viscosity-increasing substance for the continuous phase. In addition, it is not described that modified organogels can also be obtained in combination of an ethylene oxide/propylene oxide-containing O/W emulsifier with an ethylene oxide/propylene oxide-containing or else ethylene oxide/propylene oxide-containing-free W/O emulsifier which, following the addition of water, can be converted to low viscosity microemulsions and, with the subsequent addition of a crosslinker, to O/W microemulsion gels or O/W emulsions.

It is also not described that modified organogels can also be obtained in combination of an ethylene oxide/propylene oxide-containing O/W emulsifier with an ethylene oxide/propylene oxide-containing or else ethylene oxide/propylene oxide-containing-free W/O emulsifier which, following the addition of water and a crosslinker, can be converted directly to O/W microemulsion gels or O/W emulsions.

WO 0056366 describes microemulsion gels which are based on a polymeric hydrogel, such as carrageenan, xanthan gum (page 9, lines 23-32). The method of crosslinking phospholipid-containing microemulsions or emulsions is not disclosed.

In addition, WO 00610988 describes phospholipid-containing compositions which are thickened by polysaccharides, gums and polyacrylamides to give emulsions and transparent gels. The process according to the invention for the crosslinking of phospholipid-containing microemulsions or emulsions is not described. Hydrophobically modified polyethylene glycols, for example, are not described as suitable crosslinkers either.

In addition, lecithin-containing gel compositions are also known which contain alcohol. Such gel-like preparations are often liposomal gels and not O/W microemulsion gels or crosslinked emulsions, which can be ascertained easily from the lack of an oil phase in such preparations.

These objects are achieved according to the invention. The invention provides a process for preparing crosslinked microemulsion gels or crosslinked O/W emulsions of the oil-in-water type, comprising a water phase and an oil phase, which is composed essentially of difficultly volatile constituents, comprising at least one phospholipid and at least one oil-in-water emulsifier and optionally a W/O emulsifier, and if desired further auxiliaries, additives and/or active ingredients and at least one crosslinker,
a) obtainable by adding the water phase with its constituents to the oil phase with its constituents, in particular the phospholipid, the O/W emulsifier and optionally the W/O emulsifier, and where one crosslinker or two or more crosslinkers are added to the water phase or the oil phase or to both phases, where an increase in viscosity arises and the gels are obtained, and upon further addition of the water phase microemulsion gels or crosslinked O/W emulsions arise, or
b) obtainable by adding the water phase with its constituents to the oil phase with its constituents, in particular the phospholipid, the O/W emulsifier and optionally the W/O emulsifier, where a viscosity increase arises intermediately and low-viscosity phospholipid microemulsions or low-viscosity O/W emulsions are obtained which are then converted to microemulsion gels or crosslinked O/W emulsions by adding a crosslinker or two or more crosslinkers.

### BRIEF DESCRIPTION OF THE DRAWINGS

Having thus described the invention in general terms, reference will now be made to the accompanying drawings, wherein

FIGS. 1 and 2 are drawings that illustrate the backbone of a water-soluble or water-dispersible crosslinker, where the branching points represent hydrophobic groups (symbolized by rectangles) bonded covalently to the polymer.

### DETAILED DESCRIPTION OF THE PREFERRED EMBODIMENTS

Advantageously, the water phase is metered into or added dropwise to the oil phase until there is an increase in the viscosity, or a gel forms, and then the remaining water phase is metered in. The phospholipid is advantageously dissolved in the oil phase (if necessary at elevated temperature). It is, however, also possible to dissolve the phospholipid in the oil at room temperature. The O/W emulsifier and optionally the W/O emulsifier can be added directly to the oil phase or only at the gel formation stage or following preparation of the “pure” phospholipid organogel. The water phase can be added at room temperature or at elevated temperature.

The addition of the crosslinker is also not problematic and can take place in all preparation steps.

The droplets of the discontinuous oil phase are joined together by one or more crosslinker substances. The crosslinker structure is characterized by at least one hydrophilic region which has an expansion which is suitable for bridging the distance between the microemulsion or macroemulsion droplets, and by at least one hydrophobic region, in particular at least two hydrophobic regions, which are able to hydrophobically interact with the microemulsion or macroemulsion droplets. If only one hydrophobic region is present, gels or emulsions may arise in which entanglements of the hydrophilic domains of two or more polymers arise. In this way too, a gel or a crosslinked emulsion is obtained.

Here, it is equally advantageous if the crosslinker substance forms an independent gel network in which the microemulsion or macroemulsion droplets are then held by hydrophobic interaction (so-called associative thickeners are then present), or whether the network is held together as a result of the crosslinking with the microemulsion or macroemulsion droplets at points of intersection in the network.

The droplet diameters of the preparations according to the invention are preferably in the ranges specified at the beginning.

The crosslinker substances (one or more) used advantageously according to the invention generally follow structure schemes as follows: 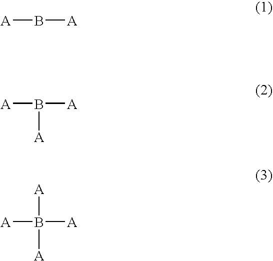
where B symbolizes a hydrophilic region of the particular crosslinker molecule, and A is in each case hydrophobic regions whose chemical nature may differ even within one molecule.

But also structure schemes such as 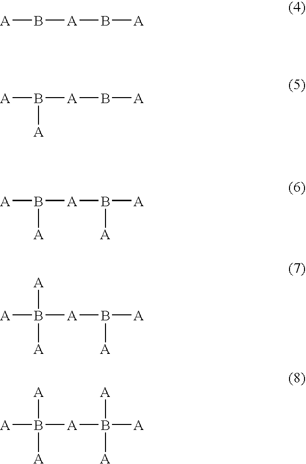
and analogously formed, yet more complex structures are definitely within the scope of the invention presented here.

The scope of the invention presented here likewise includes structure schemes as follows: 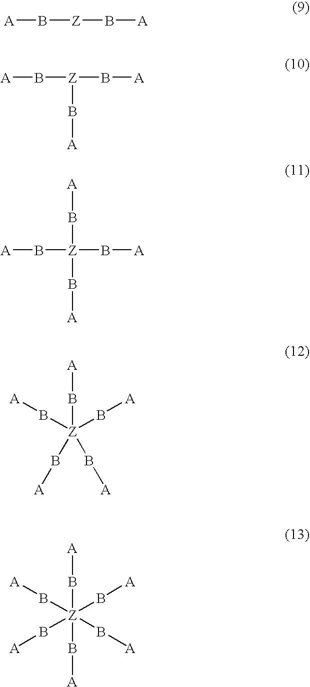
where Z is a central unit which may be hydrophilic or hydrophobic and generally consists of an oligofunctional or polyfunctional molecular radical.

Crosslinkers with a higher degree of branching are of course also within the scope of the present invention.

For example, Z in scheme (10) can consist of a glyceryl radical whose three OH functions merge into the regions B, which for their part may, for example, represent polyoxyethylene chains of equal or unequal length, and whose terminal OH group are esterified with a longer-chain fatty acid. Partial substitution on glycerol is also conceivable, as a result of which structures may form which correspond to scheme (9).

The hydrophilic groups B can advantageously be chosen such that the crosslinker overall is soluble in water or at least dispersible in water, in which case the hydrophobic moiety of groups A should then be overcompensated.

For structure scheme (1), the following more specific structure schemes may, for example, be obeyed: 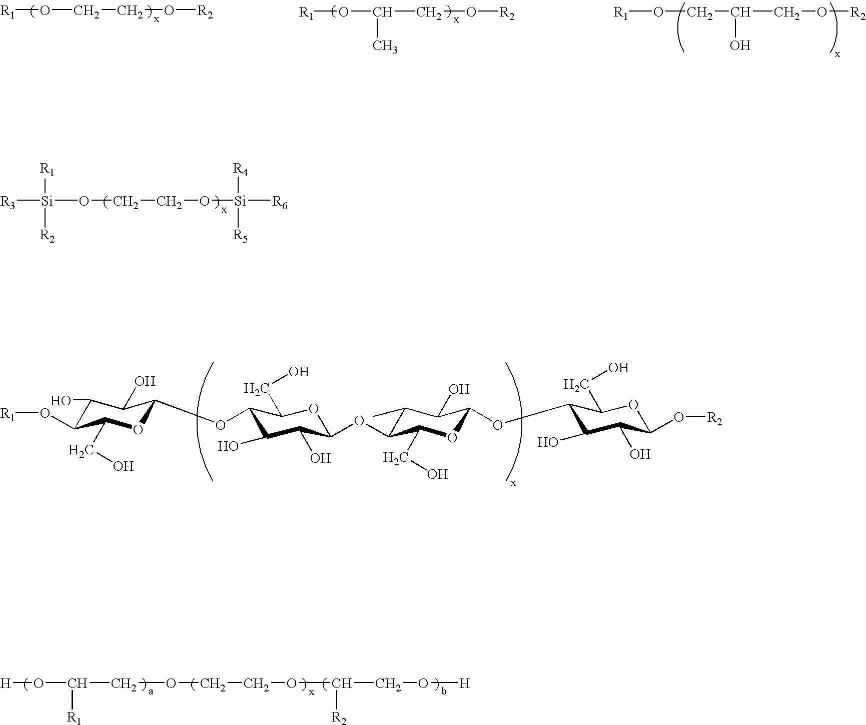
where R₁, R₂, R₃, R₄, R₅ and R₆, independently of one another, may be branched or unbranched, saturated or unsaturated, cyclic or chain-like aliphatic, aromatic or heteroaromatic radicals, for example branched or unbranched or cyclic alkyl or alkanoyl radicals, aryl or aroyl radicals which are unsubstituted or substituted by alkyl or aryl substituents, or else alkylated or arylated organylsilyl radicals. x here is numbers which allow the overall molecule to be soluble or at least dispersible in water, typically chosen from the range greater than 10, advantageously from the range 20-300. a and b are numbers which are chosen depending on x such that the overall molecule has at least adequate solubility or dispersibility in water. In individual cases, for example when the crosslinker is chosen from the group of derivatized polysaccharides, x may also assume even essentially higher values than 300, even several million. This is known per se to the person skilled in the art and requires no further explanation.

For structure scheme (2), the following more specific structure schemes may, for example, be obeyed: 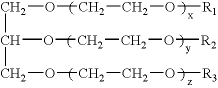
where R₁, R₂ and R₃, independently of one another, may be branched or unbranched, saturated or unsaturated, cyclic or chain-like aliphatic, aromatic or heteroaromatic radicals, for example branched or unbranched or cyclic alkyl or alkanoyl radicals, aryl or aroyl radicals which are unsubstituted or substituted by alkyl or aryl substituents, or else alkylated or arylated organylsilyl radicals. x, y and z here, independently of one another, are numbers which allow the overall molecule to be soluble or at least dispersible in water, typically chosen from the range greater than 10, advantageously from the range 20-300.

Partial substitution is also conceivable here, where one or more of the indices x, y or z can assume the value zero and one or more of the radicals R₁, R₂ or R₃ can represent hydrogen atoms.

For structure scheme (3), the following more specific structure schemes may, for example, be obeyed: 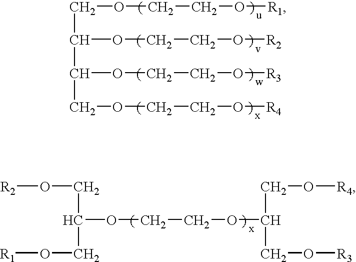
where R₁, R₂, R₃ and R₄, independently of one another, may be branched or unbranched, saturated or unsaturated, cyclic or chain-like aliphatic, aromatic or heteroaromatic radicals, for example branched or unbranched or cyclic alkyl or alkanoyl radicals, aryl or aroyl radicals which are unsubstituted or substituted by alkyl or aryl substituents, or else alkylated or arylated organylsilyl radicals. u, v, w and x here are, independently of one another, numbers which allow the overall molecule to be soluble or at least dispersible in water, typically chosen from the range greater than 10, advantageously from the range 20-300.

Here too it is of course possible for partial substitution to be conceivable, in which case one or more of the indices u, v, w, x can assume the value zero, and one or more of the radicals R₁, R₂, R₃ or R₄ can represent hydrogen atoms. The substances naturally convert to other structure schemes.

For structure scheme (9), the following more specific structure schemes, for example, may be obeyed: 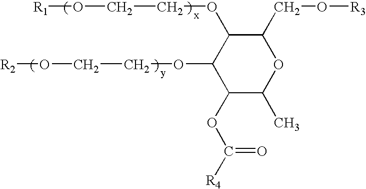
where R₁, R₂, R₃ and R₄, independently of one another, may be branched or unbranched, saturated or unsaturated, cyclic or chain-like aliphatic, aromatic or heteroaromatic radicals, for example branched or unbranched or cyclic alkyl or alkanoyl radicals, aryl or aroyl radicals which are unsubstituted or substituted by alkyl or aryl substituents, or else alkylated or arylated organylsilyl radicals. x and y here, independently of one another, are numbers which allow the overall molecule to be soluble or at least dispersible in water, typically chosen from the range greater than 10, advantageously from the range 20-300.

For structure scheme (10), the following more specific structure schemes, for example, may be obeyed: 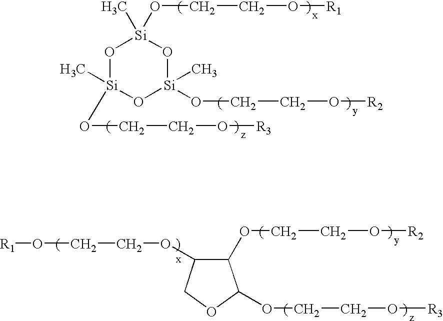
where R₁, R₂ and R₃, independently of one another, may be branched or unbranched, saturated or unsaturated, cyclic or chain-like aliphatic, aromatic or heteroaromatic radicals, for example branched or unbranched or cyclic alkyl or alkanoyl radicals, aryl or aroyl radicals which are unsubstituted or substituted by alkyl or aryl substituents, or else alkylated or arylated organylsilyl radicals. x, y and z here, independently of one another, are numbers which allow the overall molecule to be soluble or at least dispersible in water, typically chosen from the range greater than 10, advantageously from the range 20-300.

For structure scheme (11), the following more specific structure scheme, for example, may be obeyed: 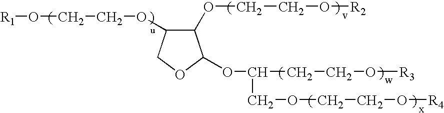
where R₁, R₂, R₃ and R₄, independently of one another, may be branched or unbranched, saturated or unsaturated, cyclic or chain-like aliphatic, aromatic or heteroaromatic radicals, for example branched or unbranched or cyclic alkyl or alkanoyl radicals, aryl or aroyl radicals which are unsubstituted or substituted by alkyl or aryl substituents, or else alkylated or arylated organylsilyl radicals. u, v, w and x here are, independently of one another, numbers which allow the overall molecule to be soluble or at least dispersible in water, typically chosen from the range greater than 10, advantageously from the range 20-300. k, l, m and n here, independently of one another, may be numbers from 0 to 50.

For structure scheme (12), the following more specific structure scheme, for example, may be obeyed: 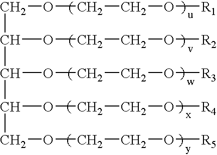
where R₁, R₂, R₃, R₄ and R₅, independently of one another, may be branched or unbranched, saturated or unsaturated, cyclic or chain-like aliphatic, aromatic or heteroaromatic radicals, for example branched or unbranched or cyclic alkyl or alkanoyl radicals, aryl or aroyl radicals which are unsubstituted or substituted by alkyl or aryl substituents, or else alkylated or arylated organylsilyl radicals. u, v, w, x and y here, independently of one another, are numbers which allow the overall molecule to be soluble or at least dispersible in water, typically chosen from the range greater than 10, advantageously from the range 20-100.

For structure scheme (13), the following more specific structure scheme, for example, may be obeyed: 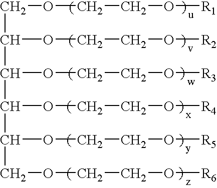
where R₁, R₂, R₃, R₄, R₅ and R₆, independently of one another, may be branched or unbranched, saturated or unsaturated, cyclic or chain-like aliphatic, aromatic or heteroaromatic radicals, for example branched or unbranched or cyclic alkyl or alkanoyl radicals, aryl or aroyl radicals which are unsubstituted or substituted by alkyl or aryl substituents, or else alkylated or arylated organylsilyl radicals. u, v, w, x, y and z here, independently of one another, are numbers which allow the overall molecule to be soluble or at least dispersible in water, typically chosen from the range greater than 10, advantageously from the range 20-1000.

In some cases, it is also advantageous to modify the structure schemes described above such that branching arises again at the end of the crosslinker molecule, for example in such a way as is realized in the group of so-called dendrimers.

Particularly suitable crosslinkers which have proven useful are those chosen from the group
of polyethylene glycol ethers of the general formula R—O—(—CH₂—CH₂—O—)ₙ—R′, where R and R′, independently of one another, are branched or unbranched alkyl, aryl or alkenyl radicals and n is a number greater than 100,
of etherified fatty acid ethoxylates of the general formula R—COO—(—CH₂—CH₂—O—)ₙ—R′, where R and R′, independently of one another, are branched or unbranched alkyl, aryl or alkenyl radicals and n is a number greater than 100,
of esterified fatty acid ethoxylates of the general formula R-COO-(—CH₂—CH₂-O—)ₙ—C(O)—R′, where R and R′, independently of one another, are branched or unbranched alkyl, aryl or alkenyl radicals and n is a number greater than 100, of polypropylene glycol ethers of the general formula R—O—(—CH₂—CH(CH₃)—O—)ₙ—R′, where R and R′, independently of one another, are branched or unbranched alkyl, aryl or alkenyl radicals and n is a number greater than 100, of esterified fatty acid propoxylates of the general formula R—COO—(—CH₂—CH(CH₃)—O—)ₙ—C(O)—R′, where R and R′, independently of one another, are branched or unbranched alkyl, aryl or alkenyl radicals and n is a number greater than 100, of polypropylene glycol ethers of the general formula R—O—Xₙ—Yₘ—R′, where R and R′, independently of one another, are branched or unbranched alkyl, aryl or alkenyl radicals, where X and Y are not identical and in each case are either an oxyethylene group or an oxypropylene group and n and m, independently of one another, are numbers whose sum is greater than 100,
of etherified fatty acid propoxylates of the general formula R—COO—Xₙ—Yₘ—R′, where R and R′, independently of one another, are branched or unbranched alkyl, aryl or alkenyl radicals, where X and Y are not identical and are in each case either an oxyethylene group or an oxypropylene group and n and m, independently of one another, are numbers whose sum is greater than 100.

PEG-150 distearate, PEG 800 distearate, PEG 800 Chol₂ and PEG-150 dioleate are particularly advantageous. PEG-300 pentaerythrityl tetraisostearate, PEG-120 methylglucose dioleate, PEG-160 sorbitan triisostearate, PEG-450 sorbitol hexaisostearate and PEG-230 glyceryl triisostearate are to be used advantageously as crosslinkers. In addition, PEG-200 glyceryl palmitate is also suitable. In addition, the crosslinker from Südchemie with the name Purethix 1442 (polyether-1) is also advantageous. It is also possible to use polyurethane crosslinkers, such as Rheolate 204, 205, 208 (Rheox) or cosmetic variants modified therefrom or DW 1206B from Rhom & Haas or Serad Fx 1010, 1035 from Hüls. In addition, it is also advantageous to use mixtures of the polymers described above, for example of PEG 800 distearate and PEG-800 Chol₂.

A slightly modified way of forming microemulsion gels according to the invention consists in immobilizing the oil droplets through the use of hydrophobically modified, synthetic or natural polymers. Such polymers are sometimes also referred to as associative thickeners.

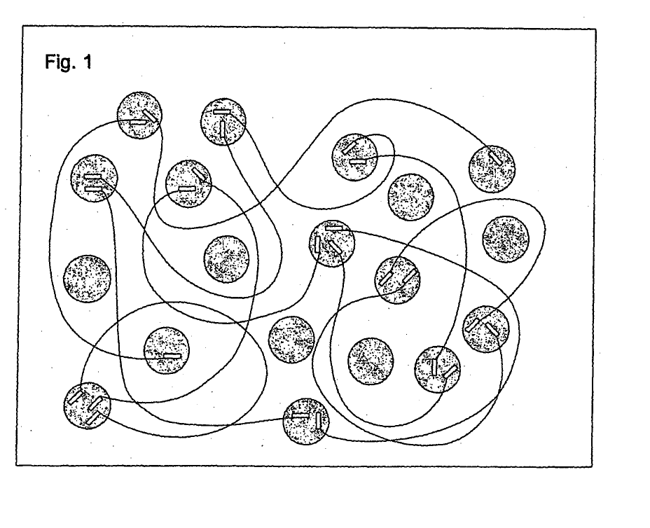

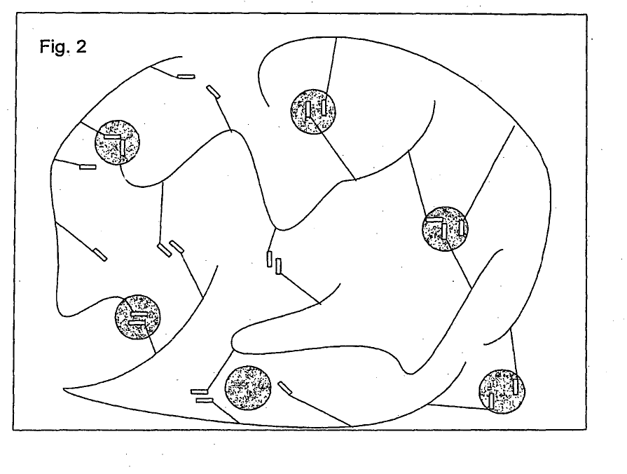

In FIGS. 1 and 2, lines show the backbone of a water-soluble or water-dispersible crosslinker, where the branching points represent hydrophobic groups bonded covalently to the polymer, symbolized here by rectangles. The hydrophobic radicals can position themselves next to each other as a result of hydrophobic interaction. Microemulsion droplets can likewise position themselves at the crosslinking points as a result of hydrophobic interactions. In this connection, it is essentially unimportant whether the hydrophobic radicals “dip in” or whether the hydrophobic radicals are in merely superficial contact with the microemulsion droplets and adhere to these to a greater or lesser degree.

It is accordingly also advantageous, particularly when the crosslinker or the crosslinkers are to be chosen from the group of associative thickeners, to choose hydrophobically substituted polysaccharide derivatives, for example hydrophobically substituted cellulose ethers, hydrophobically substituted starches, alginates, glucans, chitins, dextrans, caseinates, pectins, proteins and gums, and also polyurethanes, polyacrylamides, polyvinyl alcohols, polyacrylates, water-soluble silicone polymers and the like.

For example, cetylhydroxyethylcellulose can be used advantageously.

In some instances, it may also be advantageous if the crosslinker or crosslinkers used according to the invention has or have physiological effectiveness in the sense of a cosmetic or pharmaceutical effect. Thus, for example, the biosurfactant esters disclosed in German laid-open specification 43 44 661 can be used advantageously for the purposes of the present invention. In addition, in the case of the crosslinked emulsions and microemulsions, combinations of the above-described crosslinkers with classic polymers which are not able to crosslink due to their structure are also possible, which are referred to below as thickeners. Inorganic thickeners can also be used.

The inorganic thickener or thickeners can, for example, be chosen advantageously from the group of modified or unmodified, naturally occurring or synthetic sheet silicates. Very advantageous inorganic gel formers for the purposes of the present invention are aluminum silicates, such as the montmorillonites (bentonites, hectorites and derivatives thereof, such as quaternium-18 bentonite, quaternium-18 hectorite, stearalkonium bentonite and stearalkonium hectorite), but also magnesium aluminum silicates (Veegum® grades) and sodium magnesium silicates (Laponite® grades). Bentone® is a trade name for various neutral and chemically inert gelling agents which are constructed from long-chain, organic ammonium salts and specific montmorillonite grades.

The group of cosmetically and dermatologically relevant hydrocolloids may, for example, be:
organic, natural compounds, such as, for example, agar agar, carrageen, tragacanth, gum arabic, alginates, pectins, polyoses, guar flour, carob bean flour, starch, dextrins, gelatin, casein,
organic, modified natural substances, such as, for example, carboxymethylcellulose and other cellulose ethers, hydroxyethylcellulose and hydroxypropylcellulose and microcrystalline cellulose and the like,
organic, completely synthetic compounds, such as, for example, polyacrylic and polymethacrylic compounds, vinyl polymers, polycarboxylic acids, polyethers, polyimines, polyamides, polyurethanes,
inorganic compounds, such as, for example, polysilicic acids, clay minerals, such as montmorillonites, zeolites, silicas.

Further hydrocolloids which are advantageous according to the invention are, for example, methylcelluloses, which is the term used for the methyl ethers of cellulose. They are characterized by the following structural formula 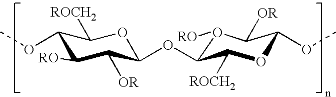
in which R may be a hydrogen or a methyl group.

Particularly advantageous for the purposes of the present invention are the cellulose mixed ethers, which are generally likewise referred to as methylcelluloses, which contain, besides a predominating content of methyl groups, additionally 2-hydroxyethyl groups, 2-hydroxypropyl groups or 2-hydroxybutyl groups. Particular preference is given to (hydroxypropyl)methylcelluloses, for example those available under the trade name Methocel® E4M from Dow Chemical Comp.

Also advantageous according to the invention is sodium carboxymethylcellulose, the sodium salt of the glycolic ether of cellulose, for which R in structural formula I may be a hydrogen and/or CH₂—COONa. Particular preference is given to the sodium carboxymethylcellulose available under the trade name Natrosol Plus 330 CS from Aqualon and also referred to as cellulose gum.

Also preferred for the purposes of the present invention is xanthan (CAS No. 11138-66-2), also called xanthan gum, which is an anionic heteropolysaccharide which is usually formed by fermentation from corn sugar and is isolated as the potassium salt. It is produced by Xanthomonas campestris and a few other species under aerobic conditions and has a molecular weight of from 2×10⁶ to 24×10⁶. Xanthan is formed from a chain having β-1,4-bonded glucose (cellulose) with side chains. The structure of the subgroups consists of glucose, mannose, glucuronic acid, acetate and pyruvate. Xanthan is the name given to the first microbial anionic heteropolysaccharide. It is produced by Xanthomonas campestris and a few other species under aerobic conditions and has a molecular weight of 2-15 10⁶. Xanthan is formed from a chain having β-1,4-bonded glucose (cellulose) with side chains. The structure of the subgroups consists of glucose, mannose, glucuronic acid, acetate and pyruvate. The number of pyruvate units determines the viscosity of the xanthan. Xanthan is produced in two-day batch cultures with a yield of 70-90%, based on carbohydrate used. In this connection, yields of 25-30 g/l are achieved. After the culture has been destroyed, work-up takes place by precipitation with, for example, 2-propanol. Xanthan is then dried and ground.

An advantageous gel former for the purposes of the present invention is also carrageen, a gel-forming extract with a similar structure to agar, from north Atlantic red algae, which belong to the Florideae (Chondrus crispus and Gigartina stellata).

The term carrageen is frequently used for the dried algae product and carrageenan for the extract thereof. The carrageen precipitated from the hot water extract of the algae is a colorless to sand-colored powder with a molecular weight range from 100 000-800 000 and a sulfate content of about 25%. Carrageen, which is very readily soluble in warm water, forms a thixotropic gel upon cooling, even if the water content is 95-98%. The rigidity of the gel is affected by the double helix structure of carrageen. In the case of carrageenan three main constituents are differentiated: the gel-forming κ fraction consists of D-galactose 4-sulfate and 3,6-anhydro-α-D-galactose, which has alternate glycoside bonds in the 1,3- and 1,4-position (by contrast, agar contains 3,6-anhydro-α-L-galactose). The nongelling λ fraction is composed of 1,3-glycosidically linked D-galactose 2-sulfate and 1,4-bonded D-galactose 2,6-disulfate radicals, and is readily soluble in cold water. τ-Carrageenan, composed of D-galactose 4-sulfate in 1,3 bond and 3,6-anhydro-α-D-galactose 2-sulfate in 1,4 bond, is both water-soluble and also gel-forming. The type of cations present (K⁺, NH₄⁺, Na⁺, Mg²⁺, Ca²⁺) also influences the solubility of the carrageens.

The use of chitosan in cosmetic preparations is known per se. Chitosan represents a partially deacylated chitin. This biopolymer has, inter alia, film-forming properties and is characterized by a silky feel on the skin. A disadvantage, however, is its severe stickiness on the skin which occurs in particular—temporarily—during application. In individual cases, corresponding preparations may not then be marketable since they are unacceptable to, or viewed negatively by, the consumer. As is known, chitosan is used, for example, in hair care. It is suitable, to a better degree than the chitin on which it is based, as a thickener or stabilizer and improves the adhesion and water resistance of polymeric films. A representative of a large number of literature references for the prior art is: H. P. Fiedler, “Lexikon der Hilfsstoffe für Pharmazie, Kosmetik und angrenzende Gebiete” [Lexicon of Auxiliaries for Pharmacy, Cosmetics and Related Fields], third edition 1989, Editio Cantor, Aulendorf, p. 293, keyword “chitosan”. It is advantageous to choose chitosans with molecular weights between 10 000 and 1 000 000, in particular those with molecular weights between 100 000 and 1 000 000 [determined by means of gel permeation chromatography].

Polyacrylates are gelling agents likewise to be used advantageously for the purposes of the present invention. Polyacrylates advantageous according to the invention are acrylate-alkyl acrylate copolymers, in particular those chosen from the group of so-called carbomers or carbopols (Carbopol® is actually a registered trademark of B. F. Goodrich Company). In particular, the acrylate-alkyl acrylate copolymer or copolymers advantageous according to the invention are characterized by the following structure: 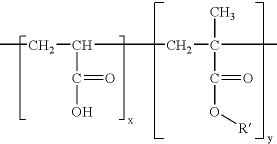
where R′ is a long-chain alkyl radical, and x and y represent numbers which symbolize the respective stoichiometric portion of each of the comonomers.

According to the invention, particular preference is given to acrylate copolymers and/or acrylate-alkyl acrylate copolymers which are available under the trade names Carbopol® 1382, Carbopol® 981 and Carbopol® 5984 from B. F. Goodrich Company, preference being given to polyacrylates from the group of carbopol grades 980, 981, 1382, 2984, 5984, and particular preference being given to Carbomer 2001.

Also advantageous are copolymers of C₁₀₋₃₀-alkylacrylates and one or more monomers of acrylic acid, of methacrylic acid or esters thereof which are crosslinked with an allyl ether of sucrose or an allyl ether of pentaerythritol.

Compounds which bear the INCI name “Acrylates/C₁₀₋₃₀ Alkyl Acrylate Crosspolymer” are advantageous. Particularly advantageous are those polymers available under the trade names Pemulen TR1 and Pemulen TR2 from B. F. Goodrich Company.

Compounds which bear the INCI name ammonium acryloyldimethyl-taurates/vinylpyrrolidone copolymers are advantageous.

According to the invention, the ammonium acryloyldimethyltaurates/vinylpyrrolidone copolymer or copolymers advantageously have the empirical formula [C₇H₁₆N₂SO₄]ₙ[C₆H₉NO]ₘ, which corresponds to the following statistical structure

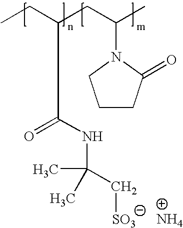

Preferred species for the purposes of the present invention are listed in the Chemical Abstracts under the registry numbers 58374-69-9, 13162-05-5 and 88-12-0 and are available under the trade name Aristoflex® AVC from Clariant GmbH.

Also advantageous are copolymers/crosspolymers comprising acryloyldimethyl taurate, such as, for example, Simugel® EG or Simugel® EG from Seppic S.A.

Further hydrocolloids to be used advantageously according to the invention are also

1. Water-soluble or -dispersible anionic polyurethanes which are advantageously obtainable from
i) at least one compound which contains two or more active hydrogen atoms per molecule,
ii) at least one diol containing acid or salt groups, and
iii) at least one diisocyanate.

Component i) is in particular diols, aminoalcohols, diamines, polyesterols, polyetherols with a number-average molecular weight of in each case up to 3000 or mixtures thereof, where up to 3 mol % of said compounds may be replaced by triols or triamines. Preference is given to diols and polyesterdiols. In particular, the component (a) comprises at least 50% by weight, based on the total weight of component (a), of a polyesterdiol. Suitable polyesterdiols are all those which are customarily used for the preparation of polyurethanes, in particular reaction products of phthalic acid and diethylene glycol, isophthalic acid and 1,4-butanediol, isophthalic acid/adipic acid and 1,6-hexanediol, and adipic acid and ethylene glycol or 5-NaSO₃-isophthalic acid, phthalic acid, adipic acid and 1,6-hexanediol.

Diols which can be used are, for example, ethylene glycol, propylene glycol, butylene glycol, neopentyl glycol, polyetherols, such as polyethylene glycols with molecular weights up to 3000, block copolymers of ethylene oxide and propylene oxide with number-average molecular weights of up to 3000 or block copolymers of ethylene oxide, propylene oxide and butylene oxide which contain the copolymerized alkylene oxide units in random distribution or in the form of blocks. Preference is given to ethylene glycol, neopentyl glycol, di-, tri-, tetra-, penta- or hexaethylene glycol. Diols which can be used are also poly(α-hydroxycarboxylic acid) diols.

Suitable aminoalcohols are, for example, 2-aminoethanol, 2-(N-methylamino)ethanol, 3-aminopropanol or 4-aminobutanol.

Suitable diamines are, for example, ethylenediamine, propylenediamine, 1,4-diaminobutane and 1,6-diaminohexane, and also α,ω-diamines which can be prepared by amination of polyalkylene oxides with ammonia.

Component ii) is, in particular, dimethylolpropanoic acid or compounds of the formulae 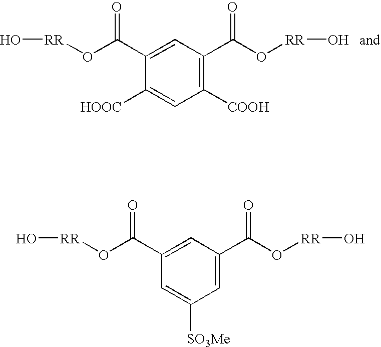
where RR is in each case a C₂-C₁₈-alkylene group and Me is Na or K.

Component iii) is in particular hexamethylene diisocyanate, isophorone diisocyanate, methyldiphenyl isocyanate (MDI) and/or tolylene diisocyanate. (calculated, in which: ηᵣ=relative viscosity (dynamic viscosity of the solution/dynamic viscosity of the solvent) and c=mass concentration of polymer in the solution (in g/cm³)).

2. Water-soluble or -dispersible cationic polyurethanes and polyureas of
a) at least one diisocyanate, which may have already been reacted beforehand with one or more compounds which contain two or more active hydrogen atoms per molecule, and
b) at least one diol, primary or secondary aminoalcohol, primary or secondary diamine or primary or secondary triamine with one or more tertiary, quaternary or protonated tertiary amino nitrogen atoms.

Preferred diisocyanates are as given above under 1). Compounds with two or more active hydrogen atoms are diols, aminoalcohols, diamines, polyesterols, polyamidediamines and polyetherols. Suitable compounds of this type are as given above under 1).

The polyurethanes are prepared as described above under 1). Charged cationic groups can be produced in the polyureas from the tertiary amino nitrogen atoms present either by protonation, e.g. with carboxylic acids such as lactic acid, or by quaternization, e.g. with alkylating agents, such as C₁- to C₄-alkyl halides or sulfates. Examples of such alkylating agents are ethyl chloride, ethyl bromide, methyl chloride, methyl bromide, dimethyl sulfate and diethyl sulfate.

These polymers and their preparation are described in more detail in DE-A-42 41 118, to the entire scope of which reference is hereby made.

3. Linear polyurethanes with carboxylate groups from
i) a 2,2-hydroxymethyl-substituted carboxylic acid of the formula 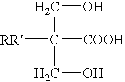
in which RR′ is a hydrogen atom or a C₁-C₂₀-alkyl group, which is used in an amount which suffices for 0.35 to 2.25 milliequivalents of carboxyl groups to be present in the polyurethane per g of polyurethane,
ii) 10 to 90% by weight, based on the weight of the polyurethane, of one or more organic compounds with not more than two active hydrogen atoms and
iii) one or more organic diisocyanates.

The carboxyl groups present in the polyurethane are, finally, at least partially neutralized with a suitable base. These polymers and their preparation are described in EP-A-619 111, to the entire scope of which reference is hereby made.

4. Carboxyl-containing polycondensation products of anhydrides of tri- or tetracarboxylic acids and diols, diamines or aminoalcohols (polyesters, polyamides or polyester amides). These polymers and their preparation are described in more detail in DE-A-42 24 761, to the entire scope of which reference is hereby made.

5. Polyacrylates and polymethacrylates, as are described in more detail in DE-A-43 14 305, 36 27 970 and 29 17 504. Reference is hereby made to these publications in their entirety.

The total amount of one or more hydrocolloids in the finished cosmetic or dermatological preparations is advantageously chosen to be less than 5% by weight, preferably between 0.01 and 1.0% by weight, based on the total weight of the preparations.

The preparations according to the invention can advantageously in each case comprise 0.001-20% by weight of one or more crosslinkers and thickeners used according to the invention. Preferably, the content of thickeners and crosslinkers is chosen in each case to be from 0.01 to 10% by weight, in particular 0.1 to 5% by weight, in each case based on the total weight of the preparations.

Here, “lecithins” also means the phospholipids, which include, for example, the following substances: phosphatidic acids, the actual lecithins, cardiolipins, lysophospholipids, lysolecithins, plasmalogens, phosphosphingolipids, sphingomyelins. Preferred substances are described below.

Phosphatidic acids are glycerol derivatives which have been esterified in the 1-sn- and 2-position with fatty acids (1-sn-position: mostly saturated, 2-position: mostly mono- or polyunsaturated), but on atom 3-sn with phosphoric acid, and are characterized by the general structural formula

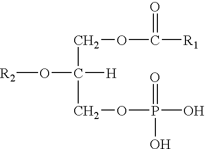

In the phosphatidic acids which occur in human or animal tissue, the phosphate radical is in most cases esterified with aminoalcohols such as choline (lecithin=3-sn-phosphatidylcholine) or 2-aminoethanol (ethanolamine) or L-serine (cephalin=3-sn-phosphatidylethanolamine or sn-phosphatidyl-L-serine), with myoinositol to give the phosphoinositides [1-(3-sn-phosphatidyl)-D-myoinositols], common in tissues, with glycerol to give phosphatidyl glycerols. Particular preference is given to lecithins (=3-sn-phosphatidylcholine).

Lecithins are characterized by the general structural formula 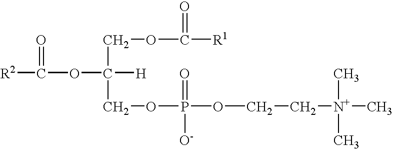
where R¹ and R² are typically unbranched aliphatic radicals having 15 or 17 carbon atoms and up to 4 cis double bonds.

Cardiolipins (1,3-bisphosphatidyl glycerols) are phospholipids of two phosphatidic acids linked via glycerol.

Lysophospholipids are obtained when an acyl radical is cleaved off by phospholipase A from phospholipids (e.g. lysolecithins).

Lysophospholipids are characterized by the general structural formula

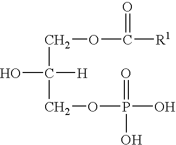

Lysolecithins, for example, are characterized by the general structural formula 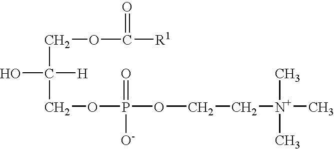
where R and R² are typically unbranched aliphatic radicals having 15 or 17 carbon atoms and up to 4 cis double bonds.

The phospholipids also include plasmalogens, in which an aldehyde (in the form of an enol ether) is bonded in the 1-position instead of a fatty acid; the O-1-sn-alkenyl compounds corresponding to the phosphatidylcholines are called, for example, hot phosphatidalcholines.

Phosphosphingolipids are based on the basic structure of sphingosine or else phytosphingosine, which are characterized by the following structural formulae

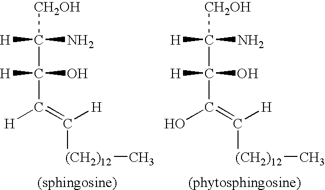

Modifications of sphingolipids are characterized, for example, by the general basic structure 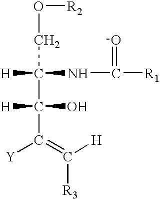
in which R₁ and R₃, independently of one another, are saturated or unsaturated, branched or unbranched alkyl radicals having 1 to 28 carbon atoms, R₂ is chosen from the group: hydrogen atom, saturated or unsaturated, branched or unbranched alkyl radicals having 1 to 28 carbon atoms, sugar radicals, phosphate groups which are unesterified or esterified with organic radicals, sulfate groups which are unesterified or esterified with organic radicals, and Y is either a hydrogen atom, a hydroxyl group or another heterofunctional radical.

Sphingophospholipids: 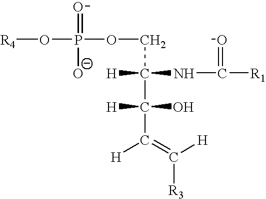
R₁ and R₃ are alkyl radicals, R₄ is an organyl radical.

Sphingomyelins are organophosphorylated sphingolipids of the type

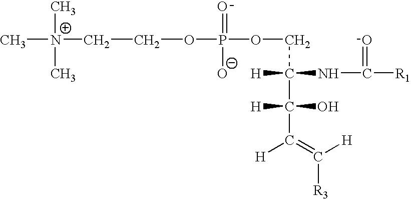

Particularly preferred phospholipids are lecithins. Types of lecithin which are to be used advantageously are chosen from crude lecithins which have been deoiled and/or fractionated and/or spray-dried and/or acetylated and/or hydrolyzed and/or hydrogenated. They are available commercially. Preference is given to soya lecithins.

Phospholipids to be used advantageously according to the invention are, for example, available commercially under the trade names Phospholipon 25 (Nattermann), Emulmetik 120 (Lucas Meyer), Sternpur E (Stern), Sternpur PM (Stern), Nathin 3KE (Stern), Phospholipon 90 (Rhône-Poulenc), Phospholipon 90H (Rhône-Poulenc), Lipoid S 100 (Lipoid). Here, it is also possible to use hydrogenated and nonhydrogenated phospholipids or corresponding combinations.

The oils and fats customary in cosmetics can be used as oil phase.

The process according to the invention permits the preparation of finely divided microemulsion gels (the droplet size is in particular about 10-200 nm; particularly advantageously 10-175 nm) or crosslinked emulsions with a large number of typical oil phases: ethers (dicaprylyl ether), carbonates (dicaprylyl carbonate), butylene glycol esters (butylene glycol caprylate, caprate), tartaric esters (Di-C12-13 alkyl tartrate), succinates (caprylic/capric diglyceryl succinate), triglycerides (caprylic/capric triglyceride), alcohols (octyldodecanol), ester oils (cetearyl isononanoate), glycerides (cocoglyceride), hydrocarbons (mineral oil, hydrogenated polydecene, isoeicosane, dioctylcyclohexane, squalane, squalene), silicone oils (cyclomethicone) and mixtures of these oil phases.

In addition, waxes may also be a constituent of the oil phase, such as, for example, methyl palmitate, cetyl palmitate, C₂₀₋₄₀-alkyl stearate, C₁₈₋₃₆-acid triglyceride.

The organogels formed as intermediates (i.e. prior to dilution with water) can be applied by the consumer, for example, as face cleansing gel, hair gel, shaving gel, make-up removal gel, aftershave gel, cleansing gel. Dilution of these gels with water then leads, depending on the O/W emulsifier, W/O emulsifier and oil phase used, to O/W microemulsions or O/W macroemulsions or corresponding gels/creams on the skin.

In addition, shower gels (foaming, nonfoaming) can be applied topically. The shower water transfers the gel on the skin into a water-continuous microemulsion or macroemulsion. The added phospholipid and further ingredients of the preparation remains on the skin (refatting). In addition, these gels can advantageously be used for the removal of skin impurities. The gels have the advantageous property of solubilizing lipid-soluble impurities in the skin. These make-up removal gels, cleansing gels, body cleansing gels may then be diluted with water by the user, the sebum being solubilized in the oil droplets, thus enabling pore-deep cleansing of the skin. At the same time, some of the phospholipid remains on the skin and thus increases the moisture content.

For the modified organogels, the following quantitative percentages by weight, in each case based on the total weight of the preparations, are preferred:

| | |
|---|---|
| Phospholipid: | 0.1-50% |
| O/W emulsifier: | 0.1-70% |
| W/O emulsifier: | 0.1-70% |
| Oil phase: | 5-90% |
| Additives for the oil phase: | 0.01-15% |
| Additives for the water phase: | 0.01-35% |
| Water | ad 100% |

For the microemulsion gels or O/W emulsions according to the invention, the following quantitative percentages by weight, in each case based on the total weight of the preparations, are preferred:

| | |
|---|---|
| Phospholipid: | 0.01-10%, in particular 0.1-5.0% |
| O/W emulsifier: | 0.01-60%, in particular 0.1-10% |
| W/O emulsifier: | 0.1-60%, in particular 0.1-10% |
| Oil phase: | 0.01-50%, in particular 0.1-30% |
| Additives for the oil phase: | 0.01-20%, in particular 0.1-15% |
| Additives for the water phase: | 0.01-80%, in particular 0.1-60% |
| Crosslinker | 0.01-70%, in particular 0.1-10% |
| Thickener (noncrosslinking): | 0.01-20%, in particular 0.1-10% |
| Water | ad 100% |

The weight ratio of phospholipid/(O/W emulsifier/W/O emulsifier) in the preparations according to the invention can vary, e.g. from 1:30 to 2:1. Preferably, the ratio of phospholipid/OW emulsifier is 1:15 to 1:1. Particularly preferably, the ratio of phospholipid/OW emulsifier is 1:6 to 1:1.3.

Here, the ratio of (phospholipid+W/O emulsifier) to O/W emulsifier can vary, e.g. 1:30 to 2:1. Preferably, the ratio of (phospholipid+W/O emulsifier) to O/W emulsifier is 1:6 to 1:1.3.

Particularly advantageous for the purposes of the present invention are O/W microemulsion gels and O/W emulsions
(a) based on microemulsions and O/W emulsions of the oil-in-water type which comprise
   a discontinuous oil phase and a continuous water phase
   optionally comprising at least one W/O emulsifier
   comprising at least one phospholipid and
   comprising at least one polyethoxylated and/or polypropoxylated O/W emulsifier
   where the polyethoxylated, polypropoxylated, or polyethoxylated and polypropoxylated O/W emulsifier; or the polyethoxylated, polypropoxylated, or polyethoxylated and polypropoxylated O/W emulsifiers; is or are chosen advantageously from the group
      of fatty alcohol ethoxylates of the general formula R—O—(—CH₂—CH₂—O—)ₙ—H, where R is a branched or unbranched alkyl, aryl or alkenyl radical and n is a number from 10 to 50
      of ethoxylated wool wax alcohols
      of polyethylene glycol ethers of the general formula R—O—(—CH₂—CH₂—O—)ₙ—R′, where R and R′, independently of one another, are branched or unbranched alkyl or alkenyl radicals and n is a number from 10 to 80
      of fatty acid ethoxylates of the general formula R—COO—(—CH₂—CH₂—O—)ₙ—H, where R is a branched or unbranched alkyl or alkenyl radical and n is a number from 10 to 40,
      of etherified fatty acid ethoxylates of the general formula R—COO—(—CH₂—CH₂—O—)ₙ—R′, where R and R′, independently of one another, are branched or unbranched alkyl or alkenyl radicals and n is a number from 10 to 80,
      of esterified fatty acid ethoxylates of the general formula R—COO—(—CH₂—CH₂—O—)ₙ—C(O)—R′, where R and R′, independently of one another, are branched or unbranched alkyl or alkenyl radicals and n is a number from 10 to 80,
      of polyethylene glycol glycerol fatty acid esters of saturated and/or unsaturated, branched and/or unbranched fatty acids and a degree of ethoxylation between 3 and 50,
      of ethoxylated sorbitan esters with a degree of ethoxylation of from 3 to 100
      of cholesterol ethoxylates with a degree of ethoxylation between 3 and 50,
      of ethoxylated triglycerides with a degree of ethoxylation between 3 and 150,
      of alkyl ether carboxylic acids of the general formula R—O—(—CH₂—CH₂—O—)ₙ—CH₂—COOH or cosmetically or pharmaceutically acceptable salts thereof, where R is a branched or unbranched alkyl or alkenyl radical having 5-30 carbon atoms and n is a number from 5 to 30,
      of polyoxyethylene sorbitol fatty acid esters based on branched or unbranched alkanoic or alkenoic acids and having a degree of ethoxylation of from 5 to 100, for example of the sorbeth type,
      of alkyl ether sulfates or the acids on which these sulfates are based of the general formula R—O—(—CH₂—CH₂—O—)ₙ—SO₃—H with cosmetically or pharmaceutically acceptable cations, where R is a branched or unbranched alkyl or alkenyl radical having 5-30 carbon atoms and n is a number from 1 to 50
      of fatty alcohol propoxylates of the general formula R—O—(—CH₂—CH(CH₃)—O—)ₙ—H, where R is a branched or unbranched alkyl or alkenyl radical and n is a number from 10 to 80,
      of polypropylene glycol ethers of the general formula R—O—(—CH₂—CH(CH₃)—O—)ₙ—R′, where R and R′, independently of one another, are branched or unbranched alkyl or alkenyl radicals and n is a number from 10 to 80
      of propoxylated wool wax alcohols,
      of etherified fatty acid propoxylates of the general formula R—COO—(—CH₂—CH(CH₃)—O—)ₙ—R′, where R and R′, independently of one another, are branched or unbranched alkyl or alkenyl radicals and n is a number from 10 to 80,
      of esterified fatty acid propoxylates of the general formula R—COO—(—CH₂—CH(CH₃)—O—)ₙ—C(O)R′, where R and R′, independently of one another, are branched or unbranched alkyl or alkenyl radicals and n is a number from 10 to 80,
      of fatty acid propoxylates of the general formula R—COO—(—CH₂—CH(CH₃)—O—)ₙ—H, where R is a branched or unbranched alkyl or alkenyl radical and n is a number from 10 to 80,
      of polypropylene glycol glycerol fatty acid esters of saturated and/or unsaturated, branched and/or unbranched fatty acids and a degree of propoxylation between 3 and 80
      of propoxylated sorbitan esters with a degree of propoxylation from 3 to 100
      of cholesterol propoxylates with a degree of propoxylation from 3 to 100
      of propoxylated triglycerides with a degree of propoxylation from 3 to 100
      of alkyl ether carboxylic acids of the general formula R—O—(—CH₂—CH(CH₃)—O—)ₙ—CH₂—COOH, or cosmetically or pharmaceutically acceptable salts thereof, where R is a branched or unbranched alkyl or alkenyl radical and n is a number from 3 to 50,
      of alkyl ether sulfates or the acids on which these sulfates are based of the general formula R—O—(—CH₂—CH(CH₃)—O—)ₙ—SO₃—H with cosmetically or pharmaceutically acceptable cations, where R is a branched or unbranched alkyl or alkenyl radical having 5-30 carbon atoms and n is a number from 1 to 50,
      of fatty alcohol ethoxylates/propoxylates of the general formula R—O—Xₙ—Yₘ—H, where R is a branched or unbranched alkyl or alkenyl radical, where X and Y are not identical and are in each case either an oxyethylene group or an oxypropylene group and n and m, independently of one another, are numbers from 5 to 50,
      of polypropylene glycol ethers of the general formula R—O—Xₙ—Yₘ—R′, where R and R′, independently of one another, are branched or unbranched alkyl or alkenyl radicals, where X and Y are not identical and are in each case either an oxyethylene group or an oxypropylene group and n and m, independently of one another, are numbers from 5 to 100,
      of etherified fatty acid propoxylates of the general formula R—COO—Xₙ—Yₘ—R′, where R and R′, independently of one another, are branched or unbranched alkyl or alkenyl radicals, where X and Y are not identical and in each case are either an oxyethylene group or an oxypropylene group and n and m, independently of one another, are numbers from 5 to 100,
      of fatty acid ethoxylates/propoxylates of the general formula R—COO—Xₙ—Yₘ—H, where R is a branched or unbranched alkyl or alkenyl radical, where X and Y are not identical and are in each case either an oxyethylene group or an oxypropylene group and n and m, independently of one another, are numbers from 5 to 50,
      of water-dispersible silicone emulsifiers
   the W/O emulsifiers (one or more), which are present if desired, can preferably be chosen from the group
      of fatty alcohol ethoxylates of the general formula R—O—(—CH₂—CH₂—O—)ₙ—H, where R is a branched or unbranched alkyl, aryl or alkenyl radical and n is a number from 1 to 10
      of polyethylene glycol ethers of the general formula R—O—(—CH₂—CH₂—O—)ₙ—R′, where R and R′, independently of one another, are branched or unbranched alkyl or alkenyl radicals and n is a number from 1 to 30
      of fatty acid ethoxylates of the general formula R—COO—(—CH₂—CH₂—O—)ₙ—H, where R is a branched or unbranched alkyl or alkenyl radical and n is a number from 1 to 20,
      of esterified fatty acid ethoxylates of the general formula R—COO—(—CH₂—CH₂—O—)ₙ—C(O)—R′, where R and R′, independently of one another, are branched or unbranched alkyl or alkenyl radicals and n is a number from 1 to 20,
      of esterified fatty acid ethoxylates of the general formula R—COO—(—CH₂—CH₂—O—)ₙ—C(O)—R′, where R and R′, independently of one another, are branched or unbranched alkyl, hydroxyalkyl or alkenyl radicals and n is a number from 1 to 40,
      of etherified fatty acid ethoxylates of the general formula R—COO—(—CH₂—CH₂—O—)ₙ—R′, where R and R′, independently of one another, are branched or unbranched alkyl or alkenyl radicals and n is a number from 1 to 4
      of fatty alcohol propoxylates of the general formula R—O—(—CH₂—CH(CH₃)—O—)ₙ—H, where R is a branched or unbranched alkyl or alkenyl radical and n is a number from 1 to 30,
      of polyoxyethylene sorbitan fatty acid esters based on branched or unbranched alkanoic or alkenoic acids and having a degree of ethoxylation of from 1 to 10
      of cholesterol ethoxylates with a degree of ethoxylation between 1 and 10,
      of ethoxylated glycerides with a degree of ethoxylation of from 1 to 30
      of ethoxylated triglycerides with a degree of ethoxylation between 1 and 30,
      of monoglycerol ethers of the type R—O—CH₂—C(H)OH—CH₂OH, where R is a branched or unbranched alkyl, aryl or alkenyl radical and
      of monoglycerol esters of the type RC(O)OCH₂—C(H)OH—CH₂OH, where R is a branched or unbranched alkyl, hydroxyalkyl, aryl or alkenyl radical
      of diglycerol esters of the type RC(O)OCH₂—C(H)OH—CH₂OC(O)R′, where R and R′, independently of one another, are branched or unbranched alkyl, hydroxyalkyl or alkenyl radicals and n is a number from 1 to 30 or,
      of polyglycerolmono- or di- or polyesters, where the fatty acids, independently of one another, are branched or unbranched alkyl, hydroxyalkyl or alkenyl radicals,
      of pentaerythritol esters, where the fatty acids, independently of one another, are branched or unbranched alkyl, hydroxyalkyl or alkenyl radicals,
      of propylene glycol esters, where the fatty acids, independently of one another, are branched or unbranched alkyl, hydroxyalkyl or alkenyl radicals,
      of sorbitan esters, where the fatty acids, independently of one another, are branched or unbranched alkyl, hydroxyalkyl or alkenyl radicals,
      of fatty alcohols R—OH and fatty acids RCOOH, where R is a branched or unbranched alkyl or alkenyl radical,
      of silicone emulsifiers, such as, for example, dimethicone copolyol, alkyl dimethicone copolyol (cetyl dimethicone copolyol), alkyl methicone copolyols (lauryl methicone copolyol), octyl dimethicone ethoxy glucoside
      of methylglucose esters, where the fatty acids, independently of one another, are branched or unbranched alkyl, hydroxyalkyl or alkenyl radicals.

The total emulsifier content is preferably 0.01 to 20% by weight, based on the total weight of the preparation.

In particular, it is advantageous if the polyethoxylated, polypropoxylated, or polyethoxylated and polypropoxylated O/W emulsifier; or the polyethoxylated, polypropoxylated, or polyethoxylated and polypropoxylated O/W emulsifiers; is or are chosen from the group
of fatty alcohol ethoxylates of the general formula R—O—(—CH₂—CH₂—O—)ₙ—H, where R is a branched or unbranched alkyl or alkenyl radical having 5-30 carbon atoms and n is a number from 10 to 25 —of ethoxylated wool wax alcohols with HLB values of 11-16,
of polyethylene glycol ethers of the general formula R—O—(—CH₂—CH₂—O—)ₙ—R′, where R and R′, independently of one another, are branched or unbranched alkyl or alkenyl radicals having 5-30 carbon atoms and n is a number from 10 to 25,
of fatty acid ethoxylates of the general formula R—COO—(—CH₂—CH₂—O—)ₙ—H, where R is a branched or unbranched alkyl or alkenyl radical having 5-30 carbon atoms and n is a number from 10 to 25,
of etherified fatty acid ethoxylates of the general formula R—COO—(—CH₂—CH₂—O—)ₙ—R′, where R and R′, independently of one another, are branched or unbranched alkyl or alkenyl radicals having 5-30 carbon atoms and n is a number from 10 to 50,
of esterified fatty acid ethoxylates of the general formula R—COO—(—CH₂—CH₂—O—)ₙ—C(O)—R′, where R and R′, independently of one another, are branched or unbranched alkyl or alkenyl radicals having 5-30 carbon atoms and n is a number from 10 to 50,
of polyethylene glycol glycerol fatty acid esters of saturated and/or unsaturated, branched and/or unbranched fatty acids having 6 to 26 carbon atoms and a degree of ethoxylation between 3 and 40
of ethoxylated sorbitan esters with a degree of ethoxylation of from 3 to 30
of cholesterol ethoxylates with HLB values of 11-16,
of ethoxylated triglycerides with HLB values of 11-16,
of alkyl ether carboxylic acids of the general formula R—O—(—CH₂—CH₂—O—)ₙ—CH₂—COOH or cosmetically or pharmaceutically acceptable salts thereof, where R is a branched or unbranched alkyl or alkenyl radical having 5-30 carbon atoms and n is a number from 10 to 20,
of polyoxyethylene sorbitol fatty acid esters based on branched or unbranched alkanoic or alkenoic acids and having a degree of ethoxylation of from 10 to 80, for example of the sorbeth type,
of alkyl ether sulfates or the acids on which these sulfates are based of the general formula R—O—(—CH₂—CH₂—O—)ₙ—SO₃—H with cosmetically or pharmaceutically acceptable cations, where R is a branched or unbranched alkyl or alkenyl radical having 5-30 carbon atoms and n is a number from 3 to 30,
of fatty alcohol propoxylates of the general formula R—O—(—CH₂—CH(CH₃)—O—)ₙ—H, where R is a branched or unbranched alkyl or alkenyl radical having 5-30 carbon atoms and n is a number from 10 to 30,
of polypropylene glycol ethers of the general formula R—O—(—CH₂—CH(CH₃)—O—)ₙ—R′, where R and R′, independently of one another, are branched or unbranched alkyl or alkenyl radicals having 5-30 carbon atoms and n is a number from 10 to 40,
of propoxylated wool wax alcohols with HLB values of 11-16,
of fatty acid propoxylates of the general formula R—COO—(—CH₂—CH(CH₃)—O—)ₙ—H, where R is a branched or unbranched alkyl or alkenyl radical having 5-30 carbon atoms and n is a number from 10 to 40, —of etherified fatty acid propoxylates of the general formula R—COO—(—CH₂—CH(CH₃)—O—)ₙ—R′, where R and R′, independently of one another, are branched or unbranched alkyl or alkenyl radicals having 5-30 carbon atoms and n is a number from 10 to 30,
of esterified fatty acid propoxylates of the general formula R—COO—(—CH₂—CH(CH₃)—O—)ₙ—C(O)—R′, where R and R′, independently of one another, are branched or unbranched alkyl or alkenyl radicals having 5-30 carbon atoms and n is a number from 10 to 50,
of polypropylene glycol glycerol fatty acid esters of saturated and/or unsaturated, branched and/or unbranched fatty acids having 6 to 26 carbon atoms and a degree of propoxylation between 3 and 50
of propoxylated sorbitan esters with a degree of propoxylation from 3 to 80
of cholesterol propoxylates with HLB values of 11-16,
of propoxylated triglycerides with HLB values of 11-16,
of alkyl ether carboxylic acids of the general formula R—O—(—CH₂—CH(CH₃)—O—)ₙ—CH₂—COOH or cosmetically or pharmaceutically acceptable salts thereof, where R is a branched or unbranched alkyl or alkenyl radical having 5-30 carbon atoms and n is a number from 10 to 30,
of alkyl ether sulfates or the acids on which these sulfates are based of the general formula R—O—(—CH₂—CH(CH₃)—O—)ₙ—SO₃—H with cosmetically or pharmaceutically acceptable cations, where R is a branched or unbranched alkyl or alkenyl radical having 5-30 carbon atoms and n is a number from 1 to 30
of water-dispersible silicone emulsifiers of the type bis PEG/PPG-16/16 PEG/PPG16/16 dimethicone+caprylic/capric triglyceride (Abil Care 85).

According to the invention, the polyethoxylated and/or polypropoxylated O/W emulsifiers used are particularly advantageously chosen from the group of substances with HLB values of 11-16 if the O/W emulsifiers have saturated radicals R and R′. If the O/W emulsifiers have unsaturated radicals R and/or R′, or if isoalkyl derivatives are present, then the preferred HLB value of such emulsifiers may also be lower or higher.

It is advantageous to choose the fatty alcohol ethoxylates from the group of ethoxylated stearyl alcohols, cetyl alcohols, cetylstearyl alcohols (cetearyl alcohols). Particular preference is given to polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(21) stearyl ether (steareth-21), polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20), polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20), polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20), polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15), polyethylene glycol(12) lauryl ether (laureth-12), polyethylene glycol(12) isolauryl ether (isolaureth-12), polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol(19) cetylstearyl ether (ceteareth-19), and polyethylene glycol(20) cetylstearyl ether (ceteareth-20).

It is also advantageous to choose the fatty acid ethoxylates from the following group: polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, and polyethylene glycol(20) oleate.

The ethoxylated alkyl ether carboxylic acid or salt thereof which can be used is advantageously sodium laureth-11 carboxylate.

Sodium laureth-4 sulfate can be used advantageously as alkyl ether sulfate.

An advantageous ethoxylated cholesterol derivative which may be used is polyethylene glycol(30) cholesteryl ether. Polyethylene glycol(25) soyasterol has also proven useful.

Ethoxylated triglycerides which can be used advantageously are polyethylene glycol(60) evening primrose glycerides.

It is also advantageous to choose the polyethylene glycol glycerol fatty acid esters from the group consisting of polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate/caprinate, polyethylene glycol(20) glyceryl oleate, polyethylene glycol(20) glyceryl isostearate, and polyethylene glycol(18) glyceryl oleate/cocoate.

It is likewise favorable to choose the sorbitan esters from the group consisting of polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

Abil Care 85 may be chosen as silicone emulsifier.

W/O emulsifiers that are optional but advantageous according to the invention are: fatty alcohols having 8 to 30 carbon atoms, monoglycerol esters of saturated and/or unsaturated, branched and/or unbranched alkanecarboxylic acids or hydroxyalkanecarboxylic acids with a chain length of from 8 to 24, in particular 12-18, carbon atoms, diglycerol esters of saturated and/or unsaturated, branched and/or unbranched alkanecarboxylic acids or hydroxyalkanecarboxylic acids with a chain length of from 8 to 24, in particular 12-18, carbon atoms, monoglycerol ethers of saturated and/or unsaturated, branched and/or unbranched alcohols with a chain length of from 8 to 24, in particular 12-18, carbon atoms, diglycerol ethers of saturated and/or unsaturated, branched and/or unbranched alcohols with a chain length of from 8 to 24, in particular 12-18, carbon atoms, propylene glycol esters of saturated and/or unsaturated, branched and/or unbranched alkanecarboxylic acids or hydroxyalkanecarboxylic acids with a chain length of from 8 to 24, in particular 12-18, carbon atoms, and sorbitan esters of saturated and/or unsaturated, branched and/or unbranched alkanecarboxylic acids or hydroxyalkanecarboxylic acids with a chain length of from 8 to 24, in particular 12-18, carbon atoms.

Particularly advantageous W/O emulsifiers are glyceryl monostearate, glyceryl monoisostearate, glyceryl linoleate, triglycerol diisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprinate, glyceryl monocaprylate, polyglyceryl-3 methylglucose distearate, PEG-45/dodecyl glycol copolymer, methoxy-PEG-22-dodecyl glycol copolymer, methylglucose sesquistearate, polyglyceryl-2 dipolyhydroxystearate, cetyl dimethicone copolyols, alkyl methicone copolyols, alkyl dimethicohe ethoxy glucosides, PEG-40 sorbitan perisostearate, PEG-30 dipolyhydroxystearate.

According to the invention, it is possible to keep the total content of emulsifiers to less than 15% by weight, based on the total weight of the microemulsion gels and O/W emulsions according to the invention. It is preferred to keep the total content of emulsifiers to less than 10% by weight, in particular to less than 8% by weight, based on the total weight of the microemulsion gels.

Skin moisturizers which can be used advantageously are glycerol, chitosan, Fucogel, 2-methylpropanediol, polyethylene glycol, lactic acid, propylene glycol, dipropylene glycol, butylene glycol, mannitol, acids and salts thereof, such as sodium pyrolidonecarboxylic acid, hyaluronic acid, amino acids, urea, electrolytes, preferably sodium, potassium, magnesium and calcium salts. Glycerol on its own and in combination with one of the abovementioned moisturizers is particularly advantageous.

It can be shown that microemulsion gels and O/W emulsions containing skin-moisturizing ingredients have excellent properties with regard to the moisturization, smoothing and reduction in flakiness of the skin. This is generally known only of classic creams or lotions, but not of transparent gels.

The oil phase of the preparations according to the invention is advantageously chosen from the group of esters of saturated and/or unsaturated, branched and/or unbranched alkanecarboxylic acids with a chain length of from 3 to 30 carbon atoms and saturated and/or unsaturated, branched and/or unbranched alcohols with a chain length of from 3 to 30 carbon atoms; from the group of esters of aromatic carboxylic acids and saturated and/or unsaturated, branched and/or unbranched alcohols with a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

The oil phase can also advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids with a chain length of from 8 to 24, in particular 12-18, carbon atoms. The fatty acid triglycerides can, for example, be chosen advantageously from the group of synthetic, semisynthetic and natural oils, e.g. olive oil, sunflower oil, soybean oil, peanut oil, rapeseed oil, almond oil, palm oil, coconut oil, palm kernel oil and the like.

For example, dicaprylyl carbonate, butylene glycol caprylate/caprate, di-C12-13 alkyl tartrate, caprylic/capric diglyceryl succinate, caprylic/capric triglyceride, octyldodecanol, cetearyl isononanoate, cocoglyceride, mineral oil, hydrogenated polydecene, isoeicosane, dioctylcyclohexane, squalane, squalene, C₁₂₋₁₅-alkyl benzoate and mixtures of these oil phases are advantageous.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

In addition, waxes may also be a constituent of the oil phase, such as, for example, methyl palmitate, cetyl palmitate, C₂₀₋₄₀-alkyl stearate, C₁₈₋₃₆-acid triglyceride. In such cases, the O/W microemulsions according to the invention can also be produced, if appropriate, as microdispersions of solid wax particles. Any desired mixtures of such oil and wax components can also be used advantageously for the purposes of the present invention.

The oil phase can advantageously also have a content of cyclic or linear silicone oils or consist entirely of such oils, although it is preferred to use an additional content of other oil phase components apart from the silicone oil or the silicone oils. Cyclomethicone (octamethylcyclotetrasiloxane) is advantageously used as silicone oil to be used according to the invention. However, other silicone oils can also be used advantageously for the purposes of the present invention, for example hexamethylcyclotrisiloxane, polydimethylsiloxane, poly(methylphenylsiloxane). Mixtures of cyclomethicone and isotridecyl isononanoate, and cyclomethicone and 2-ethylhexyl isostearate are also particularly advantageous.

The microemulsion gels according to the invention advantageously comprise electrolytes, in particular one or more salts with the following anions: chlorides, and also inorganic oxo element anions, of these in particular sulfates, carbonates, phosphates, borates and aluminates. Electrolytes based on organic anions can also be used advantageously, e.g., lactates, acetates, benzoates, propionates, tartrates, citrates and others. Comparable effects can also be achieved by ethylenediaminetetraacetic acid and salts thereof.

The cations of the salts used are preferably ammonium, alkylammoniun, alkali metal, alkaline earth metal, magnesium, iron or zinc ions. It goes without saying that only physiologically safe electrolytes should be used in cosmetics. Specific medicinal applications of the microemulsions according to the invention may, on the other hand, at least in principle, necessitate the use of electrolytes which should not be used without medical supervision.

Particular preference is given to sodium and potassium chloride, sodium and potassium bromide, magnesium and calcium chloride, magnesium and calcium bromide, zinc sulfate and mixtures thereof. Salt mixtures as occur in the natural salt of the Dead Sea are likewise advantageous. All of these salts are advantageous since they stimulate endogenous lipid synthesis.

The concentration of the electrolyte or of the electrolytes should be, for example, about 0.1-10.0% by weight, particularly advantageously about 0.3-8.0% by weight, based on the total weight of the preparation.

The microemulsion gels or emulsions according to the invention also advantageously contribute to skin smoothing, in particular when they are provided with one or more substances which promote skin smoothing.

The preparations described below may be microemulsion gels or O/W emulsions according to the invention.

If the microemulsion gels or O/W emulsions according to the invention are bases for cosmetic deodorants/antiperspirants, then all of the customary active ingredients may be used advantageously, for example odor concealers, such as customary perfume constituents, odor absorbers, for example the sheet silicates described in the patent laid-open specification DE-P 40 09 347, and of these, in particular, montmorillonite, kaolinite, ilite, beidellite, nontronite, saponite, hectorite, bentonite, smectite, and also, for example, zinc salts of ricinoleic acid. Antimicrobial agents are likewise suitable to be incorporated into the microemulsions according to the invention. Advantageous substances are, for example, 2,4,4′-trichloro-2′-hydroxydiphenyl ether (Irgasan), 1,6-di(4-chlorophenylbiguanido)hexane (chlorhexidine), 3,4,4′-trichlorocarbanilide, quaternary ammonium compounds, oil of cloves, mint oil, oil of thyme, triethyl citrate, farnesol (3,7,11-trimethyl-2,6,10-dodecatrien-1-ol), and the active agents described in the patent laid-open specifications DE-37 40 186, DE-39 38 140, DE-42 04 321, DE-42 29 707, DE-42 29 737, DE-42 37 081, DE-43 09 372, DE-43 24 219.

The customary antiperspirant active ingredients can likewise be used advantageously in the microemulsions according to the invention, in particular astringents, for example basic aluminum chlorides.

The cosmetic deodorants according to the invention can be in the form of aerosols, i.e. preparations which can be sprayed from aerosol containers, squeezable bottles or by a pump device, or in the form of liquid compositions which can be applied by means of roll-on devices, but also in the form of microemulsions which can be applied from normal bottles and containers.

Suitable propellants for cosmetic deodorants according to the invention which can be sprayed from aerosol containers are the customary known readily volatile, liquefied propellants, for example hydrocarbons (propane, butane, isobutane), which can be used on their own or in a mixture with one another. Compressed air can also be used advantageously.

The person skilled in the art is of course aware that there are propellant gases that are nontoxic per se that would in principle be suitable for the present invention but which nevertheless have to be dispensed with due to an unacceptable impact on the environment or other accompanying circumstances, in particular chlorofluorocarbons (CFCs).

Moreover, it has surprisingly been found that, when using propellants that are soluble in the oil phase, for example, customary propane/butane mixtures, the O/W microemulsions or emulsions according to the invention are not simply sprayed as aerosol droplets, but develop to give finely bubbled, rich foams as soon as such systems containing such propellants experience a pressure release.

Such after-foaming preparations are therefore likewise regarded as being advantageous embodiments of the present invention with an independent inventive step.

When using propellants that are insoluble in the oil phase, the preparations according to the invention are sprayed as aerosol droplets.

Also favorable are those cosmetic and dermatological preparations that are present in the form of a sunscreen. Preferably, besides the active ingredient combinations according to the invention, these additionally comprise at least one UVA filter substance and/or at least one UVB filter substance and/or at least one inorganic pigment.

It is, however, also advantageous for the purposes of the present invention to create those cosmetic and dermatological preparations whose main purpose is not protection against sunlight but nevertheless have a content of UV protection substances. Thus, for example, UV-A and/or UV-B filter substances are usually incorporated into day creams.

Preparations according to the invention can advantageously comprise substances which absorb UV radiation in the UVB region, the total amount of the filter substances being, for example, 0.1% by weight to 30% by weight, preferably 0.5 to 10% by weight, in particular 1 to 6% by weight, based on the total weight of the preparations.

The UVB filters may be oil-soluble or water-soluble. Examples of oil-soluble substances are:
3-benzylidenecamphor and derivatives thereof, e.g. 3-(4-methylbenzylidene)camphor,
4-aminobenzoic acid derivatives, preferably 2-ethylhexyl 4-(dimethylamino)benzoate, amyl 4-(dimethylamino)benzoate;
esters of cinnamic acid, preferably 2-ethylhexyl 4-methoxycinnamate, isopentyl 4-methoxycinnamate;
esters of salicylic acid, preferably 2-ethylhexyl salicylate, 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di(2-ethylhexyl) 4-methoxybenzalmalonate;
triazines, such as 2,4,6-trianilino(p-carbo-2′-ethyl-1′-hexyloxy)-1,3,5-triazine, dioctylbutamidotriazone (Uvasorb HEB, Sigma 3V), triazines (triazoles) under the trade name Tinosorb M and S (Ciba), Uvinul T 150
dibenzoylmethane derivatives [for example 4-(tert-butyl)-4′-methoxydibenzoylmethane],
1,4-di(2-oxo-10-sulfo-3-bornylidenemethyl)benzene and/or salts thereof and/or 2,4-bis{[4-(2-ethylhexyloxy)-2-hydroxylphenyl}-6-(4-methoxyphenyl)-1,3,5-triazine, in each case individually or in any combinations with one another.

The water-soluble substances are advantageously:
2-phenylbenzimidazole-5-sulfonic acid, phenylene-1,4-bis(2-benzimidazyl)-3,3′-5,5′-tetrasulfonic acid and/or salts thereof, e.g. sodium, potassium or triethanolammonium salts, and the sulfonic acid itself
sulfonic acid derivatives of benzophenones, preferably 2-hydroxy-4-methoxybenzophenone-5-sulfonic acid and its salts;
sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and its salts.

A further photoprotective filter substance to be used advantageously according to the invention is ethylhexyl 2-cyano-3,3-diphenylacrylate (octocrylene), which is available from BASF under the name Uvinul® N 539.

It may also be of considerable advantage to use polymer-bound or polymeric UV filter substances in preparations according to the present invention, in particular those as are described in WO-A-92/20690.

In addition, it may in some instances be advantageous to incorporate, in accordance with the invention, further UV-A and/or UV-B filters into cosmetic or dermatological preparations, for example certain salicylic acid derivatives, such as 4-isopropylbenzyl salicylate, 2-ethylhexyl salicylate (=Octyl salicylate), homomenthyl salicylate.

The list of UVB filters mentioned that can be used according to the invention is, of course, not intended to be limiting.

The invention also provides the combination of a UVA filter according to the invention with a UVB filter or a cosmetic or dermatological preparation according to the invention which also comprises a UVB filter.

It may also be advantageous to incorporate UVA filters that are usually present in cosmetic and/or dermatological preparations into preparations according to the invention. Such substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. Preparations that comprise these combinations are also provided by the invention. It is possible to use the same amounts of UVA filter substances that have been specified for UVB filter substances.

Cosmetic and/or dermatological preparations according to the invention may also comprise inorganic pigments which are customarily used in cosmetics for protecting the skin against UV rays. These are oxides of titanium, zinc, iron, zirconium, silicon, manganese, aluminum, cerium and mixtures thereof, and also modifications in which the oxides are the active agents. They are particularly preferably pigments based on titanium dioxide. The amounts specified for the abovementioned combinations may be used.

A surprising property of the present invention is that preparations according to the invention are very good vehicles for cosmetic or dermatological active ingredients into the skin, advantageous active ingredients being antioxidants that can protect the skin against oxidative stress.

According to the invention, the preparations advantageously comprise one or more antioxidants. Favorable, but nevertheless optional, antioxidants to be used are all antioxidants which are customary or suitable for cosmetic and/or dermatological applications. In this connection, it is advantageous to use antioxidants as the sole class of active ingredient when, for example, a cosmetic or dermatological application is at the forefront, such as controlling the oxidative stress of the skin. It is, however, also favorable to provide the preparations according to the invention with a content of one or more antioxidants if the preparations are to serve another purpose, e.g. as deodorants or sunscreens.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and also salts thereof, dilauryl thiodipropionate, distearyl thiodipropionate, thiodipropionic acid and derivatives thereof (esters, ethers, peptides, lipids, nucleotides, nucleosides and salts), and also sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, ferulic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of these said active ingredients that are suitable according to the invention.

For the purposes of the present invention, water-soluble antioxidants may be used particularly advantageously.

A surprising property of the preparations according to the invention is that they are very good vehicles for cosmetic or dermatological active ingredients into the skin, preferred active ingredients being antioxidants that can protect the skin against oxidative stress. Preferred antioxidants here are vitamin E and derivatives thereof, and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or the antioxidants, it is advantageous to choose their particular concentrations from the range from 0.001 to 10% by weight, based on the total weight of the formulation.

If vitamin A or vitamin A derivatives, or carotenes or derivatives thereof are the antioxidant or the antioxidants, it is advantageous to choose their particular concentrations from the range from 0.001 to 10% by weight, based on the total weight of the formulation.

According to the invention, the active ingredients (one or more compounds) can also very advantageously be chosen from the group of lipophilic active ingredients, in particular from the following group:

acetylsalicylic acid, atropine, azulene, hydrocortisone and derivatives thereof, e.g. hydrocortisone-17 valerate, vitamins, e.g. ascorbic acid and derivatives thereof, vitamins of the B and D series, very favorably vitamin B₁, vitamin B₁₂, vitamin D₁, but also bisabolol, unsaturated fatty acids, namely the essential fatty acids (often also called vitamin F), in particular gamma-linolenic acid, oleic acid, eicosapentanoic acid, docosahexanoic acid and derivatives thereof, chloramphenicol, caffeine, prostaglandins, thymol, camphor, extracts or other products of a vegetable and animal origin, e.g. evening primrose oil, borage oil or currant seed oil, fish oils, cod-liver oil and also ceramides and ceramide-like compounds, etc.

It is also advantageous to choose the active ingredients from the group of refatting substances, for example purcellin oil, Eucerit® and Neocerit®.

The active ingredient or ingredients is/are particularly advantageously chosen from the group of NO synthase inhibitors, particularly if the preparations according to the invention are to be used for the treatment and prophylaxis of the symptoms of intrinsic and/or extrinsic skin aging and for the treatment and prophylaxis of the harmful effects of ultraviolet radiation on the skin. A preferred NO synthase inhibitor is nitroarginine.

The active ingredient or ingredients is/are further advantageously chosen from the group which includes catechins and bile esters of catechins and aqueous or organic extracts from plants or parts of plants which have a content of catechins or bile esters of catechins, such as, for example, the leaves of the theaceae plant family, in particular of the species Camellia sinensis (green tea). Their typical ingredients (such as, for example, polyphenols or catechins, caffeine, vitamins, sugars, minerals, amino acids, lipids) are particularly advantageous.

Catechins are a group of compounds which are to be regarded as hydrogenated flavones or anthocyanidins and are derivatives of “catechin” (catechol, 3,3′,4′,5,7-flavanpentol, 2-(3,4-dihydroxyphenyl)chroman-3,5,7-triol). Epicatechin ((2R,3R)-3,3′,4′,5,7-flavanpentol) is also an advantageous active ingredient for the purposes of the present invention.

Also advantageous are plant extracts with a content of catechins, in particular extracts of green tea, such as, for example, extracts from leaves of the plants of the species Camellia spec., very particularly of the tea types Camellia sinensis, C. assamica, C. taliensis and C. irrawadiensis and hybrids of these with, for example, Camellia japonica.

Preferred active ingredients are also polyphenols and catechins from the group consisting of (−)-catechin, (+)-catechin, (−)-catechin gallate, (−)-gallocatechin gallate, (+)-epicatechin, (−)-epicatechin, (−)-epicatechin gallate, (−)-epigallocatechin, (−)-epigallocatechin gallate.

Flavone and its derivatives (also often collectively called “flavones”) are also advantageous active ingredients for the purposes of the present invention. They are characterized by the following basic structure (substitution positions are shown):

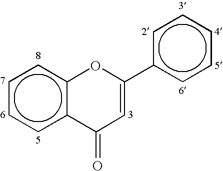

Some of the more important flavones which can also preferably be used in preparations according to the invention are given in the table below:

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones are usually in glycosylated form.

According to the invention, the flavonoids are preferably chosen from the group of substances of the generic structural formula 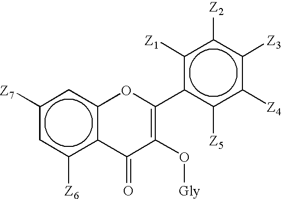
where Z₁ to Z₇, independently of one another, are chosen from the group consisting of H, OH, alkoxy and hydroxyalkoxy groups, where the alkoxy and hydroxyalkoxy groups can be branched or unbranched and may have 1 to 18 carbon atoms, and where Gly is chosen from the group of mono- and oligoglycoside radicals.

According to the invention, the flavonoids can, however, also be chosen advantageously from the group of substances of the generic structural formula 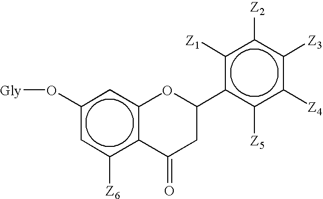
where Z₁ to Z₆, independently of one another, are chosen from the group consisting of H, OH, alkoxy and hydroxyalkoxy groups, where the alkoxy and hydroxyalkoxy groups may be branched or unbranched and have 1 to 18 carbon atoms, and where Gly is chosen from the group of mono- and oligoglycoside radicals.

Preferably, such structures can be chosen from the group of substances of the generic structural formula 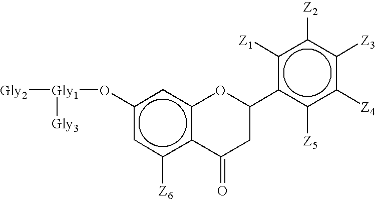
where Gly₁, Gly₂ and Gly₃, independently of one another, are monoglycoside radicals. Gly₂ and Gly₃ may also, individually or together, represent saturations by hydrogen atoms.

Preferably, Gly₁, Gly₂ and Gly₃, independently of one another, are chosen from the group of hexosyl radicals, in particular the rhamnosyl radicals and glucosyl radicals. However, other hexosyl radicals, for example allosyl, altrosyl, galactosyl, gulosyl, idosyl, mannosyl and talosyl, can also be used advantageously in some circumstances. It may also be advantageous according to the invention to use pentosyl radicals.

Advantageously, Z₁ to Z₅, independently of one another, are chosen from the group consisting of H, OH, methoxy, ethoxy and 2-hydroxyethoxy, and the flavone glycosides have the structure

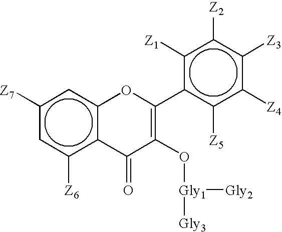

The flavone glycosides according to the invention are particularly advantageously from the group given by the following structure: 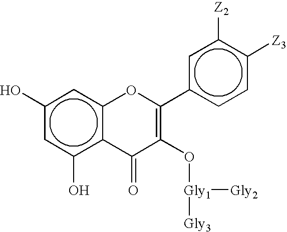
where Gly₁, Gly₂ and Gly₃, independently of one another, are monoglycoside radicals or. Gly₂ and Gly₃ may also, individually or together, represent saturations by hydrogen atoms.

Preferably, Gly₁, Gly₂ and Gly₃ are chosen independently of one another from the group of hexosyl radicals, in particular the rhamnosyl radicals and glucosyl radicals. However, other hexosyl radicals, for example allosyl, altrosyl, galactosyl, gulosyl, idosyl, mannosyl and talosyl, can also advantageously be used in some circumstances. It may also be advantageous according to the invention to use pentosyl radicals.

For the purposes of the present invention, it is particularly advantageous to choose the flavone glycoside(s) from the group consisting of α-glucosylrutin, α-glucosylmyricetin, α-glucosylisoquercitrin, α-glucosylisoquercetin and α-glucosylquercitrin.

According to the invention, particular preference is given to α-glucosylrutin.

Also advantageous according to the invention are naringin (aurantin, naringenin-7-rhamnoglucoside), hesperidin (3′,5,7-trihydroxy-4′-methoxyflavanone-7-rutinoside, hesperidoside, hesperetin-7-O-rutinoside), rutin (3,3′,4′,5,7-pentahydroxyflyvon-3-rutinoside, quercetin-3-rutinoside, sophorin, birutan, rutabion, taurutin, phytomelin, melin), troxerutin (3,5-dihydroxy-3′,4′,7-tris(2-hydroxyethoxy)flavone-3-(6-O-(6-deoxy-α-L-mannopyranosyl)-β-D-glucopyranoside)), monoxerutin (3,3′,4′,5-tetrahydroxy-7-(2-hydroxyethoxy)flavone-3-(6-O-(6-deoxy-α-L-mannopyranosyl)-β-D-glucopyranoside)), dihydrorobinetin (3,3′,4′,5′,7-pentahydroxyflavanone), taxifolin (3,3′,4′,5,7-pentahydroxyflavanone), eriodictyol-7-glucoside (3′,4′,5,7-tetrahydroxyflavanone-7-glucoside), flavanomarein (3′,4′,7,8-tetrahydroxyflavanone-7-glucoside) and isoquercetin (3,3′,4′,5,7-pentahydroxyflavanone-3-(β-D-glucopyranoside).

It is also advantageous to choose the active ingredient(s) from the group of ubiquinones and plastoquinones.

Ubiquinones are characterized by the structural formula 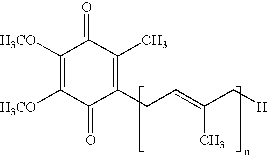
and are the most widespread and thus the most investigated bioquinones. Ubiquinones are referred to, depending on the number of isoprene units linked in the side chain, as Q-1, Q-2, Q-3 etc., or, according to the number of carbon atoms, as U-5, U-10, U-15 etc. They preferably arise with certain chain lengths, e.g. in some microorganisms and yeasts where n=6. In most mammals including man, Q10 predominates.

Coenzyme Q10 is particularly advantageous and is characterized by the following structural formula:

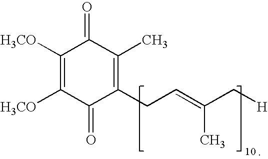

Plastoquinones have the general structural formula

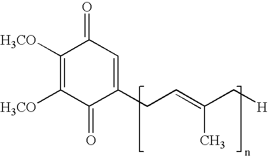

Plastoquinones differ in the number n of isoprene radicals and are referred to accordingly, e.g. PQ-9 (n=9). In addition, other plastoquinones with varying substituents on the quinone ring exist.

Creatine and/or creatinee derivatives, phosphocreatinee are also preferred active ingredients for the purposes of the present invention. Creatine is characterized by the following structure:

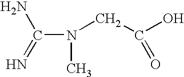

Preferred derivatives are creatinee phosphate, and creatinee sulfate, creatinee acetate, creatinee ascorbate and the derivatives esterified on the carboxyl group with mono- or polyfunctional alcohols.

A further advantageous active ingredient is L-carnitine β-hydroxy-4-(trimethylammonio)butyrobetaine]. Acylcarnitines, chosen from the group of substances of the following general structural formula 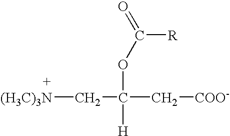
where R is chosen from the group of branched and unbranched alkyl radicals having up to 10 carbon atoms, are also advantageous active ingredients for the purposes of the present invention. Preference is given to propionylcarnitine and, in particular, acetylcarnitine. Both enantiomers (D and L form) are to be used advantageously for the purposes of the present invention. It may also be advantageous to use any enantiomer mixtures, for example a racemate of D and L form.

Further advantageous active ingredients are sericoside, pyridoxol, aminoguadine, phytochelatin, isoflavones (genistein, daidzein, daidzin, glycitin), niacin, tyrosine sulfate, dioic acid, adenosine, pyridoxine, arginine, vitamin K, biotin and aroma substances.

The list of said active ingredients and active ingredient combinations which can be used in the preparations according to the invention is not of course intended to be limiting. The active ingredients can be used individually or in any combinations with one another.

Active ingredients may be present in the preparations in the amounts of 0.0001-25% by weight, preferably 0.001-20% by weight, in particular 0.01-10% by weight, in each case based on the total weight of the preparations.

Although the use of hydrophilic active ingredients is of course also favored according to the invention, a further advantage of the microemulsions or emulsions according to the invention is that the high number of very finely divided droplets makes oil-soluble and/or lipophilic active ingredients in particular bioavailable with particularly good effectiveness.

It is also advantageous to choose the active ingredients from the group of refatting substances, for example purcellin oil, Eucerit® and Neocerit®.

It is also possible and in some instances advantageous to add washing-active surfactants to the preparations according to the invention. Aqueous cosmetic cleansing agents according to the invention or low-water or anhydrous cleansing agent concentrates intended for aqueous cleansing can comprise cationic, anionic, nonionic and/or amphoteric surfactants, for example conventional soaps, e.g. fatty acid salts of sodium, alkyl sulfates, alkyl ether sulfates, alkane- and alkylbenzenesulfonates, sulfoacetates, sulfobetaines, sarcosinates, amidosulfobetaines, sulfosuccinates, sulfosuccinic monoesters, alkyl ether carboxylates, protein-fatty acid condensates, alkylbetaines and amidobetaines, fatty acid alkanolamides, polyglycol ether derivatives.

Cosmetic preparations that are cosmetic cleansing preparations for the skin may be present in liquid or semisolid form, for example in the form of gels. They preferably comprise at least one anionic, cationic, nonionic or amphoteric surface-active substance or mixtures thereof, optionally electrolytes and auxiliaries, as are customarily used for this purpose. The surface-active substance can preferably be present in a concentration between 1 and 30% by weight in the cleansing preparations, based on the total weight of the preparations.

Cosmetic preparations which are shampoos preferably comprise at least one anionic, nonionic or amphoteric surface-active substance or mixtures thereof, optionally electrolytes and auxiliaries as are customarily used for this purpose. The surface-active substance can preferably be present in a concentration between 1 and 50% by weight in the cleansing preparations, based on the total weight of the preparations. Cetyltrimethylammonium salts, for example, are to be used advantageously.

The preparations according to the invention intended for the cleansing of hair or skin comprise, apart from the abovementioned surfactants, water and optionally the additives customary in cosmetics, for example perfume, thickeners, dyes, deodorants, antimicrobial substances, refatting agents, complexing agents and sequestrants, pearlescence agents, plant extracts, vitamins, active ingredients and the like.

Despite their oil content, the preparations according to the invention surprisingly have very good foam development, high cleansing power and have a high regenerating effect with regard to the general condition of the skin. In particular, the preparations according to the invention have a skin-smoothing effect, reduce the feeling of dryness of the skin and make the skin supple.

If the preparations according to the invention are to be used for hair care, they can comprise the customary constituents, usually, for example, film-forming polymers. Of such polymers with at least partially quaternized nitrogen groups (called below “film formers”), those which are chosen from the group of substances which carry the name “Polyquaternium” according to INCI nomenclature (international Nomenclature Cosmetic Ingredient) are preferably suitable, for example:
Polyquaternium-2 (Chemical Abstracts No. 63451-27-4, e.g. Mirapol® A-15)
Polyquaternium-5 (copolymer of acrylamide and β-methacryloxyethyltrimethylammonium methosulfate, CAS No. 26006-22-4)
Polyquaternium-6 (homopolymer of N,N-dimethyl-N-2-propenyl-2-propen-1-aminium chloride, CAS No. 26062-79-3, e.g. Merquat® 100)
Polyquaternium-7 N,N-dimethyl-N-2-propenyl-2-propen-1-aminium chloride, polymer with 2-propenamide, CAS No. 26590-05-6, e.g. Merquat® S
Polyquaternium-10 quaternary ammonium salt of hydroxyethylcellulose, CAS No. 53568-66-4, 55353-19-0, 54351-50-7, 68610-92-4, 81859-24-7, e.g. Celquat® SC-230M,
Polyquaternium-11 vinylpyrrolidone/dimethylaminoethyl methacrylate copolymer/diethyl sulfate reaction product, CAS No. 53633-54-8, e.g. Gafquat® 755N
Polyquaternium-16 vinylpyrrolidone/vinylimidazolinium methochloride copolymer, CAS No. 29297-55-0, e.g. Luviquat®) HM 552
Polyquaternium-17 CAS No. 90624-75-2, e.g. Mirapol® AD-1
Polyquaternium-19 quaternized water-soluble polyvinyl alcohol
Polyquaternium-20 water-dispersible quaternized polyvinyl octadecyl ether
Polyquaternium-21 polysiloxane-polydimethyl-dimethylammonium acetate copolymer, e.g. Abil® B 9905
Polyquaternium-22 dimethyldiallylammonium chloride/acrylic acid copolymer, CAS No. 53694-7-0, e.g. Merquat® 280
Polyquaternium-24 polymeric quaternary ammonium salt of hydroxyethylcellulose, reaction product with an epoxide substituted by lauryidimethylammonium, CAS No. 107987-23-5, e.g. Quatrisoft® LM-200
Polyquaternium-28 vinylpyrrolidone/methacrylamidopropyltrimethylammonium chloride copolymer, e.g. Gafquat® HS-100
Polyquaternium-29 e.g. Lexquat® CH
Polyquaternium-31 CAS No. 136505-02-7, e.g. Hypan® QT 100
Polyquaternium-32 N,N, N-trimethyl-2-[(2-methyl-1-oxo-2-propenyl)oxy]ethanaminium chloride, polymer with 2-propenamide, CAS No. 35429-19-7
Polyquaternium-37 CAS No. 26161-33-1
Cetyltrimethylammonium salts such as CTAB, CTAC.

Hair care preparations according to the invention advantageously comprise 0.01-5% by weight of one or more film formers, preferably 0.1-3% by weight, in particular 0.2-2% by weight, in each case based on the total weight of the preparations. Such embodiments of the preparations according to the invention care for hair which has been stripped or damaged by environmental influences, or protect against such influences. In addition, the preparations according to the invention give the hairstyle relaxed fullness and strength without having a sticky effect.

Correspondingly, depending on their formulation, the preparations according to the invention can, for example, be used as skin protection emulsion, cleansing milk, sunscreen lotion, nutrient lotion, day or night emulsion etc.

The preparations according to the invention further advantageously contribute to skin smoothing, particularly when they are provided with one or more substances which promote skin smoothing.

It is in some cases possible and advantageous to use the preparations according to the invention as bases for pharmaceutical formulations. Corresponding requirements apply mutatis mutandis to the formulation of medicinal preparations. The boundaries between pure cosmetics and pure pharmaceuticals are fluid here. According to the invention, suitable pharmaceutical active ingredients are fundamentally all classes of active ingredient, preference being given to lipophilic active ingredients. Examples are: antihistamines, antiphlogistics, antibiotics, antimycotics, active ingredients which promote circulation, keratolytics, hormones, steroids, vitamins etc.

The cosmetic and dermatological preparations according to the invention can comprise cosmetic auxiliaries as are customarily used in such preparations, e.g. preservatives, bactericides, virucides, perfumes, substances for preventing foaming, dyes, pigments which have a coloring effect, thickeners, surface-active substances, emulsifiers, softening, moisturizing and/or humectant substances, anti-inflammatory substances, medicaments, fats, oils, waxes or other customary constituents of a cosmetic or dermatological formulation, such as alcohols, polyols, polymers, foam stabilizers, electrolytes, organic solvents.

Mixtures of the abovementioned solvents are used particularly advantageously.

Further constituents which may be used are fats, waxes and other natural and synthetic fatty bodies, preferably esters of fatty acids with alcohols of low carbon number, e.g. with isopropanol, propylene glycol or glycerol, or esters of fatty alcohols with alkanoic acids of low carbon number or with fatty acids, alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

Unless stated otherwise, all amounts, percentages or parts refer to the weight of the preparations or of the particular mixture.

The examples below are intended to illustrate the present invention.

The lecithin used in the examples was phosphatidylcholine (Phospholipon 90, Nattermann).

### EXAMPLE 1

Face care gel

| | % by wt. |
|---|---|
| Lecithin | 0.500 |
| Isoceteth-20 | 2.500 |
| Glycerol isostearate | 0.500 |
| Glycerol | 5.000 |
| Cetearyl isononanoate | 2.500 |
| Tocopherol | 0.500 |
| Polyether 1 (Pure Thix TX 1442) | 1.000 |
| Preservative, antioxidant | q.s. |
| Water | ad |
| | 100.000 |

### EXAMPLE 2

Antiacne gel

| | % by wt. |
|---|---|
| Lecithin | 0.500 |
| PEG-25 stearate | 2.500 |
| Glycerol isostearate | 0.500 |
| Glycerol | 5.000 |
| Cetearyl isononanoate | 2.500 |
| Tocopherol | 0.500 |
| PEG-300 pentaerythrityl tetraisostearate | 1.000 |
| Preservative, antiacne active ingredient | q.s. |
| Water | ad |
| | 100.000 |

### EXAMPLE 3

Face care gel

| | % by wt. |
|---|---|
| Lecithin | 0.500 |
| PEG-25 sorbitan isostearate | 2.500 |
| Glycerol isostearate | 0.500 |
| Glycerol | 5.000 |
| Cetearyl isononanoate | 2.500 |
| PEG-800 distearate | 1.000 |
| Preservative | q.s. |
| Water | ad |
| | 100.000 |

### EXAMPLE 4

Face care gel

| | % by wt. |
|---|---|
| Lecithin | 0.500 |
| PEG-25 stearate | 2.500 |
| Glycerol isostearate | 0.500 |
| Glycerol | 5.000 |
| Cetearyl isononanoate | 2.500 |
| PEG-800 distearate | 1.000 |
| Preservative | q.s. |
| Water | ad |
| | 100.000 |

### EXAMPLE 5

Face care gel

| | % by wt. |
|---|---|
| Lecithin | 0.500 |
| PEG-25 stearate | 2.500 |
| Glycerol isostearate | 0.500 |
| Glycerol | 5.000 |
| Cetearyl isononanoate | 2.500 |
| Bis PEG/PPG-16/16 PEG/PPG16/16 | 0.01 |
| dimethicone + caprylic/capric | |
| triglyceride (Abil Care 85) | |
| PEG-800 distearate | 1.000 |
| Preservative | q.s. |
| Water | ad |
| | 100.000 |

### EXAMPLE 6

Hair care gel

| | % by wt. |
|---|---|
| Lecithin | 3.000 |
| Isoceteth-20 | 4.000 |
| Glycerol | 5.000 |
| Dicaprylyl ether | 5.000 |
| PEG-300 hydrogenated glyceryl palmitate | 1.00 |
| Preservative, hair care active ingredient | q.s. |
| Water | ad |
| | 100.000 |

### EXAMPLE 7

Bodycare gel

| | % by wt. |
|---|---|
| Lecithin | 3.000 |
| PEG-45 palm kernel oil glyceride | 4.000 |
| Glycerol | 5.000 |
| Dicaprylyl ether | 5.000 |
| Sericoside | 1.000 |
| Cetylhydroxyethylcellulose | 2.000 |
| Preservative | q.s. |
| Water | ad |
| | 100.000 |

### EXAMPLE 8

Base for shaving gel

| | % by wt. |
|---|---|
| Lecithin | 3.000 |
| PEG-20 sorbitan monooleate | 4.000 |
| Glycerol | 5.000 |
| Dicaprylyl carbonate | 5.000 |
| PEG-800 chol₂ | 1.000 |
| Preservative, anti-irritative active ingredient | q.s. |
| Water | ad |
| | 100.000 |

### EXAMPLE 9

Aftershave gel

| | % by wt. |
|---|---|
| Lecithin | 1.000 |
| Polyglyceryl-10 stearate | 6.000 |
| Glycerol | 5.000 |
| Dicaprylyl ether | 5.000 |
| PEG-150 distearate | 1.000 |
| Preservative, aftershave active ingredient, | q.s. |
| Panthenol | |
| Water | ad 100.000 |

### EXAMPLE 10

Face cleansing gel or antiaging gel

| | % by wt. |
|---|---|
| Lecithin | 2.000 |
| Decaglyceryl monolaurate | 5.000 |
| Glycerol | 5.000 |
| Carnithine, biotin (1:1) | 1.000 |
| Dicaprylyl ether | 5.000 |
| PEG-800 distearate | 1.000 |
| Preservative | q.s. |
| Water | ad 100.000 |

### EXAMPLE 11

Shower gel

| | % by wt. |
|---|---|
| Lecithin | 3.500 |
| PEG-20 glyceryl laurate | 3.500 |
| Glycerol | 5.000 |
| Dicaprylyl ether | 5.000 |
| PEG-150 distearate | 2.000 |
| Preservative, shower care additive | q.s. |
| Water | ad 100.000 |

### EXAMPLE 12

Deodorant/antiperspirant gel

| | % by wt. |
|---|---|
| Lecithin | 3.000 |
| PEG-20 monostearate | 4.000 |
| Glycerol | 5.000 |
| Dicaprylyl ether | 5.000 |
| Aluminum chlorohydrate | 20.00 |
| PEG-800 distearate | 1.000 |
| Preservative | q.s. |
| Water | ad 100.000 |

### EXAMPLE 13

Face care gel

| | % by wt. |
|---|---|
| Lecithin | 3.000 |
| PEG-20 glyceryl stearate | 4.000 |
| Glycerol | 5.000 |
| Creatine, lipoic acid (1:1) | 1.000 |
| Dicaprylyl ether | 5.000 |
| PEG-300 pentaerythrityl tetraisostearate | 1.000 |
| Preservative | q.s. |
| Water | ad 100.000 |

### EXAMPLE 14

Preshave gel

| | % by wt. |
|---|---|
| Lecithin | 4.000 |
| Ceteareth-12 | 3.000 |
| Glycerol | 5.000 |
| PEG-120 methyl glucose dioleate | 1.000 |
| Dicaprylyl ether | 5.000 |
| Preservative | q.s. |
| Water | ad 100.000 |

### EXAMPLE 15

Make-up removal gel

| | % by wt. |
|---|---|
| Lecithin | 2.000 |
| PEG-20 sorbitan isostearate | 5.000 |
| Glycerol | 5.000 |
| Octyl dodecanol | 5.000 |
| Lipoic acid/biotin (1:1) | 0.500 |
| Cetylhydroxylethylcellulose | 1.000 |
| Preservative | q.s. |
| Water | ad 100.000 |

### EXAMPLE 16

Perfume gel

| | % by wt. |
|---|---|
| Lecithin | 2.000 |
| PEG-20 sorbitan isostearate | 5.000 |
| Glycerol | 5.000 |
| Cetearyl isononanoate | 5.000 |
| PEG-230 glyceryl triisostearate | 1.000 |
| Preservative, perfume | q.s. |
| Water | ad 100.000 |

### EXAMPLE 17

Gel for the scalp

| | % by wt. |
|---|---|
| Lecithin | 2.000 |
| PEG-20 sorbitan isostearate | 5.000 |
| Glycerol | 5.000 |
| Dioctylcyclohexane | 5.000 |
| Urethane C₁₋₂₀-alkyl-PEG copolymer | 1.000 |
| Preservative, scalp active ingredient | q.s. |
| Water | ad 100.000 |

### EXAMPLE 18

Repellent gel containing alcohol

| | % by wt. |
|---|---|
| Lecithin | 1.000 |
| Polyglyceryl-10 stearate | 6.000 |
| Glycerol | 5.000 |
| Ethanol | 5.000 |
| Dioctylcyclohexane | 5.000 |
| PEG-150 distearate | 2.000 |
| Preservative, repellent active ingredient | q.s. |
| Water | ad 100.000 |

### EXAMPLE 19

| | % by wt. |
|---|---|
| Lecithin | 1.000 |
| Oleth-15 | 6.000 |
| Glycerol | 5.000 |
| Octyl dodecanol | 5.000 |
| Alkyl modified acrylate | 1.000 |
| Creatine/biotin | 1.000 |
| Preservative | q.s. |
| Water | ad 100.000 |

### EXAMPLE 20

Antiage gel

| | % by wt. |
|---|---|
| Lecithin | 2.000 |
| Isoceteth-20 | 5.000 |
| Glycerol | 5.000 |
| Caprylic/capric triglyceride | 5.000 |
| Carnithine/retinol (1:1) | 0.500 |
| PEG-800 distearate | 1.000 |
| Preservative | q.s. |
| Water | ad 100.000 |

### EXAMPLE 21

Antiage bodycare gel

| | % by wt. |
|---|---|
| Lecithin | 3.000 |
| PEG-45 palm kernel oil glycerides | 4.000 |
| Glycerol | 5.000 |
| Dioctylcyclohexane | 5.000 |
| Creatine ester/coenzyme Q-10 (1:1) | 0.500 |
| PEG-800 distearate/PEG-800 chol₂ (1:1) | 1.000 |
| Preservative | q.s. |
| Water | ad 100.000 |

### EXAMPLE 22

Skin whitening gel

| | % by wt. |
|---|---|
| Lecithin | 3.000 |
| PEG-45 palm kernel oil glyceride | 4.000 |
| Glycerol | 5.000 |
| Cetearyl isononanoate | 5.000 |
| Polyether 1 (Pure Thix TX 1442) | 1.000 |
| Dioic acid | 1.000 |
| Preservative | q.s. |
| Water | ad 100.000 |

### EXAMPLE 23

| | % by wt. |
|---|---|
| Lecithin | 2.000 |
| PEG-20 sorbitan monooleate | 5.000 |
| Glycerol | 5.000 |
| Octyl dodecanol | 5.000 |
| Coenzyme Q-10/lipoic acid (1:1) | 0.500 |
| Polyether 1 (Pure Thix TX 1442) | 1.000 |
| Preservative | q.s. |
| Water | ad 100.000 |

### EXAMPLE 24

| | % by wt. |
|---|---|
| Lecithin | 2.000 |
| PEG-20 sorbitan monooleate | 5.000 |
| Glycerol | 5.000 |
| Caprylic/capric triglycerides | 5.000 |
| Coenzyme Q-10/biotin (1:1) | 1.000 |
| Preservative | q.s. |
| Polyether 1 (Pure Thix TX 1442) | 1.000 |
| Water | ad 100.000 |

### EXAMPLE 25

| | % by wt. |
|---|---|
| Lecithin | 2.000 |
| PEG-20 sorbitan monooleate | 5.000 |
| Glycerol | 5.000 |
| Cetearyl isononanoate | 5.000 |
| Adenosine/lipoic acid (1:1) | 0.500 |
| PEG-120 methylglucose dioleate | 1.000 |
| Preservative | q.s. |
| Water | ad 100.000 |

### EXAMPLE 26

Shower gel

| | % by wt. |
|---|---|
| Lecithin | 0.500 |
| Lauryl ether sulfate (25%) | 40.000 |
| Glycerol | 5.000 |
| Dicaprylyl ether | 5.000 |
| Sodium chloride | 7.000 |
| PEG-120 methylglucose dioleate | 1.000 |
| Preservative | q.s. |
| Water | ad 100.000 |

### EXAMPLE 27

| | % by wt. |
|---|---|
| Lecithin | 0.500 |
| Glycerol isostearate | 0.500 |
| Glycerol | 5.000 |
| Hydrogenated polydecene | 2.500 |
| PEG-25 stearate | 2.500 |
| Polyether 1 (Pure Thix TX 1442) | 1.000 |
| Preservative | q.s. |
| Water | ad 100.000 |

### EXAMPLE 28

| | % by wt. |
|---|---|
| Lecithin | 0.500 |
| Glycerol isostearate | 0.500 |
| Glycerol | 5.000 |
| Isoeicosane | 2.500 |
| Isoceteth-20 | 2.500 |
| Polyether 1 (Pure Thix TX 1442) | 1.000 |
| Preservative | q.s. |
| Water | ad 100.000 |

### EXAMPLE 29

| | % by wt. |
|---|---|
| Lecithin | 0.500 |
| Glycerol isostearate | 0.500 |
| Glycerol | 5.000 |
| Hydrogenated polydecene | 2.500 |
| PEG-25 stearate | 2.500 |
| Polyether 1 (Pure Thix TX 1442) | 1.000 |
| Preservative | q.s. |
| Water | ad 100.000 |

### EXAMPLE 30

Face care cream

| | % by wt. |
|---|---|
| Lecithin | 0.500 |
| Isoceteth-20 | 2.500 |
| Glycerol isostearate | 0.500 |
| Glycerol | 5.000 |
| Cetearyl isononanoate | 4.500 |
| Tocopherol | 0.500 |
| Polyether 1 (Pure Thix TX 1442) | 1.000 |
| Preservative, antioxidant | q.s. |
| Water | ad 100.000 |

### EXAMPLE 31

Antiacne cream

| | % by wt. |
|---|---|
| Lecithin | 0.500 |
| PEG-25 stearate | 2.500 |
| Glycerol isostearate | 0.500 |
| Glycerol | 5.000 |
| Cetearyl isononanoate | 3.500 |
| Tocopherol | 0.500 |
| PEG-300 pentaerythrityl tetraisostearate | 1.000 |
| Preservative, antiacne active ingredient | q.s. |
| Water | ad 100.000 |

### EXAMPLE 32

Face care cream

| | % by wt. |
|---|---|
| Lecithin | 0.500 |
| PEG-25 sorbitan isostearate | 2.500 |
| Glycerol isostearate | 0.500 |
| Glycerol | 5.000 |
| Cetearyl isononanoate | 3.500 |
| PEG-800 distearate | 1.000 |
| Preservative | q.s. |
| Water | ad 100.000 |

### EXAMPLE 33

Face care gel

| | % by wt. |
|---|---|
| Lecithin | 0.500 |
| PEG-25 stearate | 2.500 |
| Glycerol isostearate | 0.500 |
| Glycerol | 5.000 |
| Cetearyl isononanoate | 4.500 |
| PEG-800 distearate | 1.000 |
| Preservative | q.s. |
| Water | ad 100.000 |

### EXAMPLE 34

Face care lotion

| | % by wt. |
|---|---|
| Lecithin | 0.500 |
| PEG-25 stearate | 2.500 |
| Glycerol isostearate | 0.500 |
| Glycerol | 5.000 |
| Cetearyl isononanoate | 4.500 |
| Bis PEG/PPG-16/16 PEG/PPG16/16 | 0.01 |
| dimethicone + caprylic/capric | |
| triglyceride (Abil Care 85) | |
| PEG-800 distearate | 1.000 |
| Preservative | q.s. |
| Water | ad 100.000 |

### EXAMPLE 35

Hair care lotion

| | % by wt. |
|---|---|
| Lecithin | 3.000 |
| Isoceteth-20 | 4.000 |
| Glycerol | 5.000 |
| Dicaprylyl ether | 7.000 |
| PEG-300 hydrogenated glyceryl palmitate | 1.00 |
| Preservative, hair care active ingredient | q.s. |
| Water | ad 100.000 |

### EXAMPLE 36

Bodycare cream/lotion

| | % by wt. |
|---|---|
| Lecithin | 3.000 |
| PEG-45 palm kernel oil glyceride | 4.000 |
| Glycerol | 5.000 |
| Dicaprylyl ether | 7.000 |
| Sericoside | 1.000 |
| Cetylhydroxyethylcellulose | 2.000 |
| Preservative | q.s. |
| Water | ad 100.000 |

### EXAMPLE 37

Base for shaving cream/lotion

| | % by wt. |
|---|---|
| Lecithin | 3.000 |
| PEG-20 sorbitan monooleate | 4.000 |
| Glycerol | 5.000 |
| Dicaprylyl carbonate | 7.000 |
| PEG-800 chol₂ | 1.000 |
| Preservative, anti-irritative active ingredient | q.s. |
| Water | ad 100.000 |

### EXAMPLE 38

Aftershave cream/lotion

| | % by wt. |
|---|---|
| Lecithin | 1.000 |
| Polyglyceryl-10 stearate | 6.000 |
| Glycerol | 5.000 |
| Dicaprylyl ether | 7.000 |
| PEG-150 distearate | 1.000 |
| Preservative, aftershave active ingredient, | q.s. |
| Panthenol | |
| Water | ad 100.000 |

### EXAMPLE 39

Face cleansing cream or antiaging cream

| | % by wt. |
|---|---|
| Lecithin | 2.000 |
| Decaglyceryl monolaurate | 5.000 |
| Glycerol | 7.000 |
| Carnithine, biotin (1:1) | 1.000 |
| Dicaprylyl ether | 5.000 |
| PEG-800 distearate | 1.000 |
| Preservative | q.s. |
| Water | ad 100.000 |

### EXAMPLE 40

Shower cream

| | % by wt. |
|---|---|
| Lecithin | 3.500 |
| PEG-20 glyceryl laurate | 3.500 |
| Glycerol | 5.000 |
| Dicaprylyl ether | 7.000 |
| PEG-150 distearate | 2.000 |
| Preservative, shower care additive | q.s. |
| Water | ad 100.000 |

That which is claimed:

1. A process for preparing a crosslinked microemulsion gel or crosslinked oil-in-water emulsion, the process comprising the steps of:
a) providing an oil phase comprising at least one phospholipid and at least one oil-in-water emulsifier, said oil phase consisting essentially of constituents having low volatility;
b) providing a water phase;
c) adding at least one crosslinker to at least one of the oil phase and the water phase;
d) adding a first portion of the water phase to the oil phase thereby increasing the viscosity of the oil phase and obtaining a gel, and
e) adding a second portion of the water phase to the gel thereby obtaining a microemulsion gel or a crosslinked oil-in-water emulsion.

2. The process as claimed in claim 1, wherein the at least one crosslinker is added to the oil phase of step a).

3. The process as claimed in claim 1, wherein the at least one crosslinker is added to the water phase of step b).

4. The process as claimed in claim 1, wherein the at least one crosslinker is added to the oil phase of step a) and the water phase of step b).

5. The process as claimed in claim 1, wherein the at least one phospholipid is selected from the group consisting of phosphatidylcholine, hydrogenated phosphatidylcholine, phosphatidylethanolamine, phosphatidylinositol, and phosphatidylserine.

6. The process as claimed in claim 1, wherein the at least one oil-in-water emulsifier includes at least one oil-in-water emulsifier selected from the group consisting of polyethoxylated emulsifiers and polypropoxylated emulsifiers.

7. The process as claimed in claim 6, wherein the oil phase in step a) further comprises at least one water-in-oil emulsifier.

8. The process as claimed in claim 1, wherein the at least one oil-in-water emulsifier includes at least one oil-in-water emulsifier selected from the group consisting of ceteth-15, ceteth-16, ceteareth-15, ceteareth-16, isoceteth-20, isosteareth-20, steareth-20, oleth-15, laureth-15, PEG-20 stearate, PEG-25 stearate, PEG-20 oleate, PEG-20 sorbitan stearate, PEG-20 sorbitan isostearate, PEG-20 sorbitan oleate, sodium laureth-11 carboxylate, sodium lauryl ether sulfate, PEG-30 cholesteryl ether, PEG-60 evening primrose glyceride, bis PEG/PPG-16/16 PEG/PPG16/16 dimethicone+caprylic/capric triglyceride, PEG-45 palm kernel oil glyceride, PEG-20 glyceryl laurate, PEG-20 glyceryl stearate, and PEG-20 glycerol isostearate.

9. The process as claimed in claim 1, wherein the oil phase in step a) further comprises at least one water-in-oil emulsifier.

10. The process as claimed in claim 9, wherein the at least one water-in-oil emulsifier includes an emulsifier selected from the group consisting of glyceryl stearate, glycerol isostearate, glyceryl linoleate, diglycerol isostearate, triglycerol diisostearate, sorbitan isostearate, propylene glycol isostearate, propylene glycol stearate, cetyl alcohol, stearyl alcohol, steareth-2, glyceryl laurate, glyceryl caprinate, glyceryl caprylate, selachyl alcohol, chimyl alcohol, PEG-5 cholesteryl ether PEG-30 dipolyhydroxystearate, polyglyceryl-3 methylglucose distearate, PEG-45/dodecyl glycol copolymer, methoxy-PEG-22-dodecyl glycol copolymer, methylglucose sesquistearate, polyglyceryl-2 dipolyhydroxystearate, cetyl dimethicone copolyols, alkyl methicone copolyols, and alkyl dimethicone ethoxy glucosides.

11. The process as claimed in claim 1, wherein the at least one crosslinkers include crosslinkers selected from the group consisting of hydrophobically substituted cellulose ethers, starches, acrylates, alginates, glucans, chitins, dextrans, caseinates, pectins, proteins and gums, polyurethanes, polyacrylamides, polyvinyl alcohols, polyacrylates, water-soluble silicone polymers, and combinations thereof.

12. The process as claimed in claim 1, wherein the at least one crosslinkers include crosslinkers selected from the group consisting of PEG-150 distearate, PEG-800 distearate, PEG-800 chol₂, PEG-150 dioleate, PEG-300 pentaerythrityl tetraisostearate, PEG-120 methyl glucose dioleate, PEG-160 sorbitan triisostearate, PEG-450 sorbitol hexaisostearate, PEG-230 glyceryl triisostearate, PEG-200 glyceryl palmitate, polyether-1, polyurethane crosslinkers Rheolate 204, 205, 208, and cetylhydroxyethylcellulose.

13. The process as claimed in claim 1, further comprising the step of adding at least one noncrosslinking thickener to one or more of the oil phase and the water phase.

14. The process as claimed in claim 1, further comprising the step of adding one or more skin moisturizing agents selected from the group consisting of glycerol, chitosan, Fucogel, 2-methylpropanediol, polyethylene glycol, lactic acid, propylene glycol, dipropylene glycol, butylene glycol, mannitol, sodium pyrolidonecarboxylic acid, hyaluronic acid, amino acids, urea, electrolytes, inorganic or organic acids, and acids and salts thereof.

15. A crosslinked microemulsion gel or crosslinked oil-in-water emulsion prepared according to the process as claimed in claim 1.

16. A process for preparing a crosslinked microemulsion gel or crosslinked oil-in-water emulsion, the process comprising the steps of:
a) providing an oil phase comprising at least one phospholipid and at least one oil-in-water emulsifier, said oil phase consisting essentially of constituents having low volatility;
b) providing a water phase;
c) adding the water phase to the oil phase thereby increasing the viscosity of the oil phase and obtaining a low-viscosity phospholipid microemulsion or a low-viscosity oil-in-water emulsion; and
d) adding at least one crosslinker to the a low-viscosity phospholipid microemulsion or a low-viscosity oil-in-water emulsion to produce the crosslinked microemulsion gel or the crosslinked oil-in-water emulsion.

17. The process as claimed in claim 16, wherein the at least one phospholipid is selected from the group consisting of phosphatidylcholine, hydrogenated phosphatidylcholine, phosphatidylethanolamine, phosphatidylinositol, and phosphatidylserine.

18. The process as claimed in claim 16, wherein the at least one oil-in-water emulsifier includes at least one oil-in-water emulsifier selected from the group consisting of polyethoxylated emulsifiers and polypropoxylated emulsifiers.

19. The process as claimed in claim 18, wherein the oil phase in step a) further comprises at least one water-in-oil emulsifier.

20. The process as claimed in claim 16, wherein the at least one oil-in-water emulsifier includes at least one oil-in-water emulsifier selected from the group consisting of ceteth-15, ceteth-16, ceteareth-15, ceteareth-16, isoceteth-20, isosteareth-20, steareth-20, oleth-15, laureth-15, PEG-20 stearate, PEG-25 stearate, PEG-20 oleate, PEG-20 sorbitan stearate, PEG-20 sorbitan isostearate, PEG-20 sorbitan oleate, sodium laureth-11 carboxylate, sodium lauryl ether sulfate, PEG-30 cholesteryl ether, PEG-60 evening primrose glyceride, bis PEG/PPG-16/16 PEG/PPG16/16 dimethicone+caprylic/capric triglyceride, PEG-45 palm kernel oil glyceride, PEG-20 glyceryl laurate, PEG-20 glyceryl stearate, and PEG-20 glycerol isostearate.

21. The process as claimed in claim 16, wherein the oil phase in step a) further comprises at least one water-in-oil emulsifier.

22. The process as claimed in claim 21, wherein the at least one water-in-oil emulsifier includes an emulsifier selected from the group consisting of glyceryl stearate, glycerol isostearate, glyceryl linoleate, diglycerol isostearate, triglycerol diisostearate, sorbitan isostearate, propylene glycol isostearate, propylene glycol stearate, cetyl alcohol, stearyl alcohol, steareth-2, glyceryl laurate, glyceryl caprinate, glyceryl caprylate, selachyl alcohol, chimyl alcohol, PEG-5 cholesteryl ether PEG-30 dipolyhydroxystearate, polyglyceryl-3 methylglucose distearate, PEG-45/dodecyl glycol copolymer, methoxy-PEG-22-dodecyl glycol copolymer, methylglucose sesquistearate, polyglyceryl-2 dipolyhydroxystearate, cetyl dimethicone copolyols, alkyl methicone copolyols, and alkyl dimethicone ethoxy glucosides.

23. The process as claimed in claim 16, wherein the at least one crosslinkers include crosslinkers selected from the group consisting of hydrophobically substituted cellulose ethers, starches, acrylates, alginates, glucans, chitins, dextrans, caseinates, pectins, proteins and gums, polyurethanes, polyacrylamides, polyvinyl alcohols, polyacrylates, water-soluble silicone polymers, and combinations thereof.

24. The process as claimed in claim 16, wherein the at least one crosslinkers include crosslinkers selected from the group consisting of PEG-150 distearate, PEG-800 distearate, PEG-800 chol₂, PEG-150 dioleate, PEG-300 pentaerythrityl tetraisostearate, PEG-120 methyl glucose dioleate, PEG-160 sorbitan triisostearate, PEG-450 sorbitol hexaisostearate, PEG-230 glyceryl triisostearate, PEG-200 glyceryl palmitate, polyether-1, polyurethane crosslinkers Rheolate 204, 205, 208, and cetylhydroxyethylcellulose.

25. The process as claimed in claim 16, wherein step d) further comprises adding at least one noncrosslinking thickener with the at least one crosslinker.

26. The process as claimed in claim 16, further comprising the step of adding one or more skin moisturizing agents selected from the group consisting of glycerol, chitosan, Fucogel, 2-methylpropanediol, polyethylene glycol, lactic acid, propylene glycol, dipropylene glycol, butylene glycol, mannitol, sodium pyrolidonecarboxylic acid, hyaluronic acid, amino acids, urea, electrolytes, inorganic or organic acids, and acids and salts thereof.

27. A crosslinked microemulsion gel or crosslinked oil-in-water emulsion prepared according to the process as claimed in claim 16.
